(19) United States
(12) Patent Application Publication
(10) Pub. No.: US 2025/0182884 A1
(43) Pub. Date: Jun. 5, 2025

(54) Methods, Systems, and Devices for Hematologic Morphology Detection and Treatment

(71) Applicant: IDEXX Laboratories, Inc., Westbrook, ME (US)

(72) Inventors: Jeremy Hammond, Standish, ME (US); James Russell, North Yarmouth, ME (US); Heidi Peta, Edmonton (CA); Kayla Carpenter, Westbrook, ME (US)

(21) Appl. No.: 18/968,694

(22) Filed: Dec. 4, 2024

Related U.S. Application Data
(60) Provisional application No. 63/605,884, filed on Dec. 4, 2023.
(60) Provisional application No. 63/700,029, filed on Sep. 27, 2024.

(51) Int. Cl. G16H 30/40; G01N 15/10; G01N 15/14; G06N 20/00; G16H 50/20

(52) U.S. Cl. CPC G16H 30/40; G01N 15/1425; G06N 20/00; G16H 50/20; G01N 2015/1006; G01N 2015/1486; G01N 2015/1493; G01N 2015/1497

(57) ABSTRACT

A method for detecting one or more conditions in a blood sample is disclosed. The method includes (i) receiving cell data from one or more sensors communicatively coupled to the first computing device; (ii) determining via a first machine learning model diagnostic data associated with a first portion of the blood sample, wherein the diagnostic data comprises one or more identifiable parameters associated with blood cells; (iii) receiving an image of a plurality of cells of a second portion of the blood sample; (iv) determining via a second machine learning model, one or more attributes of the plurality of cells; (v) based on the determined one or more attributes of the plurality of cells, updating the one or more of the identifiable parameters; and (vi) retraining the first machine learning model using the updated one or more of the identifiable parameters.

### CROSS-REFERENCE TO RELATED APPLICATIONS

This application claims the benefit of co-pending U.S. Provisional Patent Application Ser. No. 63/605,884, filed Dec. 4, 2023 and U.S. Provisional Patent Application Ser. No. 63/700,029, filed Sep. 27, 2024, which are hereby incorporated by reference it its entirety.

### TECHNICAL FIELD

The present specification relates to hematology systems, and more particularly, to hematology analyzer systems.

### BACKGROUND

Automated hematology analysis may be performed by an analyzer utilizing flow cytometry, impedance, and/or fluorescence. This type of analyzer tends to have excellent precision and accuracy in quantitative reporting for blood samples with normal morphology. However, when morphological abnormalities are present in a blood sample, this type of analyzer is not as accurate. Accordingly, a need exists for an improved method of performing hematology analysis when morphologic abnormalities are present so that improved diagnostic outcomes and treatment plans may be realized for affected patients. Without such technological improvements to existing technologies, systems, and/or devices, many patients affected with these morphologic abnormalities may be misdiagnosed or undiagnosed, and also mistreated or untreated based on errant hematology analysis.

Accordingly, features of the present disclosure can help to address these and other issues to provide an improvement to select technical fields and methods, devices, and systems to improve hematology analysis by more accurately and efficiently diagnosing morphologic abnormalities in a patient and identifying and implementing one or more treatment plans for these diagnosed abnormalities. More specifically, features of the present disclosure help address issues within and provide improvements for select technical fields, which include for example, healthcare and patient diagnostic systems, healthcare and patient data processing systems, computer-based analysis systems, and graphical user interfaces (GUIs). These features will now be described.

### SUMMARY

In one embodiment, a method includes receiving diagnostic data from a diagnostic system, the diagnostic data including one or more identifiable parameters associated with a subject sample, the one or more identifiable parameters each having a confidence level, receiving an image of a plurality of cells from a microscopy system separate from the diagnostic system, wherein the plurality of cells are associated with the subject sample, identifying, by a processor executing an image recognition machine-learning logic on the image, one or more cells of the plurality of cells as having one or more attributes, and changing one or more of the identifiable parameters based on the identified one or more attributes.

In another embodiment, an apparatus includes a processor configured to receive diagnostic data from a diagnostic system, the diagnostic data including one or more identifiable parameters associated with a subject sample, the one or more identifiable parameters each having a confidence level, receive an image of a plurality of cells from a microscopy system separate from the diagnostic system, wherein the plurality of cells are associated with the subject sample, identify, by a processor executing an image recognition machine-learning logic on the image, one or more cells of the plurality of cells as having one or more attributes; and change one or more of the identifiable parameters based on the identified one or more attributes.

In another embodiment, a system may include a digital microscope, one or more of a flow cytometer, an impedance analyzer, or a hybrid flow cytometer/impedance analyzer, and a computing device. The flow cytometer, impedance analyzer, or hybrid flow cytometer/impedance analyzer may be configured to receive diagnostic data from a diagnostic system, the diagnostic data including one or more identifiable parameters associated with a subject sample, the one or more identifiable parameters each having a confidence level; and transfer the identifiable parameters and the confidence levels to the computing device. The digital microscope may be configured to capture an image of a plurality of cells from the flow cytometer, wherein the plurality of cells are associated with the subject sample, and transmit the captured image to the computing device. The computing device may receive the one or more identifiable parameters, the confidence levels, and the image; identify, by a processor executing an image recognition machine-learning logic on the image, one or more cells of the plurality of cells as having one or more attributes, and change one or more of the identifiable parameters based on the identified one or more attributes.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 schematically depicts an example system for performing hematology analysis, according to one or more embodiments shown and described herein;

FIG. 2 schematically depicts an example hematology analyzer of the system of FIG. 1, according to one or more embodiments shown and described herein;

FIG. 3 schematically depicts an ECU of the hematology analyzer of FIG. 2, according to one or more embodiments shown and described herein;

FIG. 4 depicts memory modules of the ECU of FIG. 3, according to one or more embodiments shown and described herein;

FIG. 5 depicts exemplary data that may be output by the hematology analyzer of FIG. 2, according to one or more embodiments shown and described herein;

FIG. 6A depicts an example size-complexity/fluorescence dot plot output by the hematology analyzer of FIG. 2 showing white blood cells of a healthy subject, according to one or more embodiments shown and described herein;

FIG. 6B depicts another example size-complexity/fluorescence dot plot output by the hematology analyzer of FIG. 2 showing red blood cells and platelets of a healthy subject, according to one or more embodiments shown and described herein;

FIG. 6C depicts another example size-complexity/florescence dot plot output by the hematology analyzer of FIG. 2 showing white blood cells exhibiting left shift, according to one or more embodiments shown and described herein;

FIG. 6D depicts another example size-complexity/fluorescence dot plot, output by the hematology analyzer of FIG. 2 showing small pathologic red blood cells, according to one or more embodiments shown and described herein;

FIG. 7 schematically depicts a morphology analyzer of the system of FIG. 1, according to one or more embodiments shown and described herein;

FIG. 8 schematically depicts an ECU of the morphology analyzer of FIG. 7, according to one or more embodiments shown and described herein;

FIG. 9 schematically depicts memory modules of the ECU of FIG. 8, according to one or more embodiments shown and described herein;

FIG. 10 schematically depicts a user interface device of the system of FIG. 1, according to one or more embodiments shown and described herein;

FIG. 11 schematically depicts memory modules of the user interface device of FIG. 10, according to one or more embodiments shown and described herein;

FIG. 12A depicts an example image that may be captured by the morphology analyzer of FIG. 7, according to one or more embodiments shown and described herein;

FIG. 12B depicts a chart displaying RBC counts from the hematology analyzer and the morphology analyzer of FIG. 1, according to one or more embodiments shown and described herein;

FIG. 12C depicts a chart of RBC ratios from the hematology analyzer and the morphology analyzer of FIG. 1, according to one or more embodiments shown and described herein;

FIG. 13A depicts example data output by the hematology analyzer of FIG. 2, according to one or more embodiments shown and described herein;

FIG. 13B depicts the example data of FIG. 13A as revised by the morphology analyzer of FIG. 7, according to one or more embodiments shown and described herein;

FIG. 14A depicts example data output by the hematology analyzer of FIG. 2, according to one or more embodiments shown and described herein;

FIG. 14B depicts the example data of FIG. 14A as revised by the morphology analyzer of FIG. 7, according to one or more embodiments shown and described herein;

FIG. 15A depicts example data output by the hematology analyzer of FIG. 2, according to one or more embodiments shown and described herein;

FIG. 15B depicts the example data of FIG. 15A as revised by the morphology analyzer of FIG. 7, according to one or more embodiments shown and described herein;

FIG. 16A depicts example data output by the hematology analyzer of FIG. 2, according to one or more embodiments shown and described herein;

FIG. 16B depicts the example data of FIG. 16A as revised by the morphology analyzer of FIG. 7, according to one or more embodiments shown and described herein;

FIG. 17 depicts a flowchart of an example method for operating the hematology analyzer of FIG. 2, according to one or more embodiments shown and described herein;

FIG. 18 depicts a flowchart of an example method for operating the morphology analyzer of FIG. 7, according to one or more embodiments shown and described herein; and

FIG. 19 depicts a flowchart of an example method for operating the user interface device of FIG. 10, according to one or more embodiments shown and described herein.

FIG. 20 depicts a flowchart of an example method, according to one or more embodiments shown and described herein.

FIG. 21 depicts a flowchart of an example method, according to one or more embodiments shown and described herein.

### DETAILED DESCRIPTION

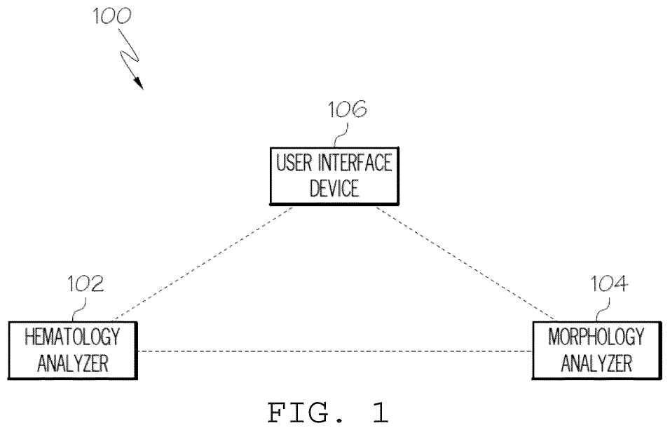

Instrumentation designed for medical or veterinary diagnostics typically perform a specified task and report information. As an example, diagnostic devices can perform hematological analysis that interrogates the structure of blood cells. For hematology analysis, a whole blood sample may be drawn into a tube containing ethylenediaminetetraacetic acid (EDTA) to preserve the cells and inhibit platelet activation and clumping. The blood sample can then be presented to analyzers for analysis. Turning now to FIG. 1, a system 100 is illustrated for performing analysis of blood samples. The system 100 comprises a hematology analyzer 102, a morphology analyzer 104, and a user interface device 106. In the embodiment depicted in FIG. 1, the hematology analyzer 104, the morphology analyzer, and the user interface device 106 are communicatively coupled to each other. As used herein, the term “communicatively coupled” means that coupled components are capable of exchanging data signals with one another such as, for example, electrical signals via conductive medium, electromagnetic signals via air, optical signals via optical waveguides, and the like.

In the illustrated example, the hematology analyzer 102 is a flow cytometer. In some examples, the hematology analyzer 102 operates based on impedance, fluorescence, flow cytometry, or the like, or any combination thereof. In examples, the morphology analyzer 104 is a digital microscope. However, some examples, the morphology analyzer 104 may be any suitable morphology analyzer for interrogating the morphology of cells. The hematology analyzer 102 and the morphology analyzer 104 generate diagnostic data comprising one or more identifiable parameters associated with a blood sample.

In the illustrated example, the user interface device 106 is a computing device that a user may use to input data, and that may receive data from the hematology analyzer 102 and the morphology analyzer 104. In embodiments, the user interface device 106 displays the received results to the user. The user interface device 106, in embodiments, is a local computing device (e.g., a desktop computer, a tablet, a smartphone, and the like), or a remote computing device (e.g., a cloud computing device). While in the embodiment depicted in FIG. 1, the user interface device 106, the hematology analyzer 102, and the morphology analyzer 104 are each communicatively coupled to one another, it should be understood that this is merely an example. In some embodiments, the hematology analyzer 102 and the morphology analyzer 104 are each communicatively coupled to the user interface device 106 individually, and are communicatively coupled to one another through the user interface device 106. In some embodiments, the hematology analyzer 102 and the morphology analyzer 104 are communicatively coupled to one another, and one of the hematology analyzer 102 or the morphology analyzer 104 is communicatively coupled to the user interface device 106, such that each of the morphology analyzer 104 and the hematology analyzer 102 are directly or indirectly communicatively coupled to the user interface device 106.

Moreover, while in the embodiment depicted in FIG. 1 the user interface device 106, the hematology analyzer 102, and the morphology analyzer 104 are shown as separate devices that are communicatively coupled to one another, it should be understood that this is merely an example. In some embodiments, the user interface device 106 is integral with one of the hematology analyzer 102 or the morphology analyzer 104. In some embodiments, the hematology analyzer 102 and the morphology analyzer 104 are integral with one another. In some embodiments, the hematology analyzer 102, the morphology analyzer 104, and the user interface device 106 are integral with one another. Moreover, while a single user interface device 106 is depicted in FIG. 1, it should be understood that the system 100 may have multiple user interface devices 106 communicatively coupled to the hematology analyzer 102 and the morphology analyzer 104.

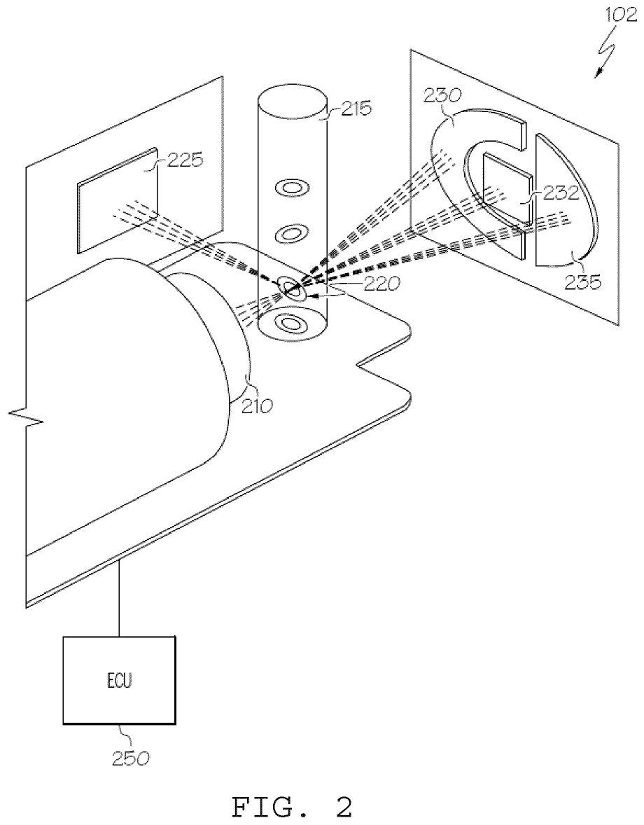

FIG. 2 illustrates a schematic view of the hematology analyzer 102 of FIG. 1. In embodiments, the hematology analyzer 102 is a flow cytometry system, which includes an energy source 210, and an electronic control unit (ECU) 250. The hematology analyzer 102, in embodiments, includes a cuvette/flow cell 215 and sensors 225, 230, 232, and 235. The energy source 210 and the sensors 225, 230, 232, and 235 are communicatively coupled to the ECU 250 such that the ECU 250 can send signals to and/or receive signals from the energy source 210 and the sensors 225, 230, 232, 235. The components are illustrated for explanatory purposes and are not drawn to scale.

In operation, as a hematology sample's constituents 220 (e.g., cells) move one cell at a time through the cuvette/flow cell 215, the energy source 210 emits a beam of energy that is oriented transverse to the axial flow of the sample's constituents 220 through the cuvette/flow cell 215. The beam of energy emitted by the energy source 210 has a central axis. In embodiments, the beam can be a focused narrow band energy beam (e.g., a LASER) or can be a broadband energy beam.

In examples, a portion of the beam from the energy source 210 that impinges upon the sample's constituents 220 (e.g., the cells) flowing in the cuvette/flow cell 215 is scattered at a right angle or substantially a right angle to the central axis of the beam of energy (side scattered energy, denoted as “SS”) and is sensed/measured by the SS sensor 225. As used herein, the term “substantially a right angle” means and includes scattered energy which is sensed/measured by SS sensor 225, even though it may not be scattered at exactly a right angle. With respect to energy scattered in the hematology systems described herein, any angle with respect to an axis means and includes such angle in any plane that includes the entire axis, without regard to the direction of the angle (e.g., 3° above an axis and 3° below an axis are both encompassed). As persons skilled in the art will understand, an infinite number of planes wholly include an axis, and an angle as used herein may be in any such plane.

In some examples, varying magnitudes of energy are scattered in all directions from each cell. As such, a magnitude signal may be received from each cell at each sensor angle. Evaluations may be performed of how these responses present together to develop algorithms to classify cells based on their scattering properties. In some examples, the extinction (EXT) sensor 232 may be used to determine absorption as energy is scattered to the various sensors, and a total of energy not transmitted to the EXT sensor 232 may define a magnitude of scattered and absorbed energy.

Another portion of the beam from the energy source 210 that impinges upon the constituents flowing in the cuvette/flow cell 215 is scattered at a much lower angle than 90° with respect to the central axis of the beam of energy. This scatter is termed “low angle forward scattered energy” or “low angle forward scattered light” (FSL) and has an angle range, for example, between approximately 1° to approximately 3° from the central axis of the beam from the energy source 210, inclusive of the endpoints, or can have another angle range that persons skilled in the art will recognize. In the illustrated embodiment, the FSL sensor 235 is oriented to capture/measure the low angle forward scatter energy and is oriented at approximately 1° to approximately 3° from the central axis of the beam of the energy source 210, inclusive of the endpoints.

In the depicted hematology analyzer 102, various other energy may be sensed/measured, and persons skilled in the art will recognize them. In embodiments, such other energy include extinction/axial energy (EXT) (e.g., from approximately 0° to approximately 0.5°, inclusive of the endpoints), which is sensed/measured by the EXT sensor 232, and “high angle forward scattered energy” or “high angle forward scattered light” (FSH) (e.g., from approximately 4° to approximately 9°, inclusive of the endpoints), which is sensed/measured by the FSH sensor 230. Such energy and angle ranges are exemplary, and other energy and other angle ranges will be recognized by persons skilled in the art. In embodiments, a time metric called time-of-flight (TOF) may be measured and analyzed. As persons skilled in the art will recognize, TOF refers to the amount of time that a sample's constituent (e.g., a cell) is interrogated by the beam from the energy source 210. TOF may be determined based on EXT energy sensed/measured by EXT sensor 232 or based on readings from the FSL sensor 235. In embodiments, fluorescence energy may be sensed/measured. The disclosure below may refer to one or more of SS, FSL, FSH, EXT, TOF and fluorescence, as examples of energy and metrics that can be used in accordance with aspects of the present disclosure. It is intended and will be understood that other flow cytometry and/or hematology system signals and metrics not expressly mentioned herein are also encompassed within the scope of the present disclosure. Furthermore, the configuration of sensors 225, 230, 232, 235 in FIG. 2 is exemplary. In embodiments, the hematology analyzer 102 may be implemented by other configurations of sensors or components different from those shown in FIG. 2.

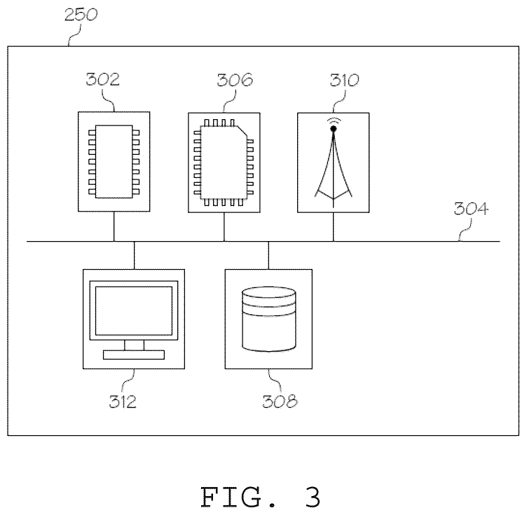

FIG. 3 schematically depicts an example configuration of the ECU 250 of FIG. 2. In the illustrated example, the ECU 250 includes one or more processors 302, a communication path 304, one or more memory modules 306, a data storage component 308, network interface hardware 310, and an output device 312.

Each of the one or more processors 302 may be any device capable of executing machine readable and executable instructions. Accordingly, each of the one or more processors 302 may be a controller, an integrated circuit, a microchip, a computer, or any other physical or cloud-based computing device local to or remote from the hematology analyzer 102 (FIG. 1). The algorithms, including the trained models, signal preprocessing, and noise removal methods discussed below, may be executed by the one or more processors 302. The one or more processors 302 are communicatively coupled to a communication path 304 that provides signal interconnectivity between various modules of the ECU 250. Accordingly, the communication path 304 may communicatively couple any number of processors 302 with one another, and allow the modules coupled to the communication path 304 to operate in a distributed computing environment. Specifically, each of the modules may operate as a node that may send and/or receive data.

The communication path 304 may be formed from any medium that is capable of transmitting a signal such as, for example, conductive wires, conductive traces, optical waveguides, or the like. In some embodiments, the communication path 304 may facilitate the transmission of wireless signals, such as WiFi, Bluetooth®, Near Field Communication (NFC) and the like. Moreover, the communication path 304 may be formed from a combination of mediums capable of transmitting signals. In one embodiment, the communication path 304 comprises a combination of conductive traces, conductive wires, connectors, and buses that cooperate to permit the transmission of electrical data signals to components such as processors, memories, sensors, input devices, output devices, and communication devices. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium.

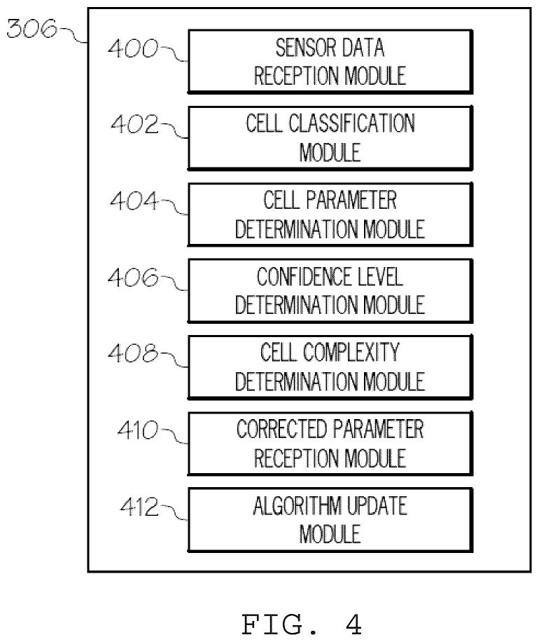

The ECU 250 includes the one or more memory modules 306 communicatively coupled to the communication path 304. The one or more memory modules 306 may comprise RAM, ROM, flash memories, hard drives, or any tangible component or device capable of storing machine readable and executable instructions such that the machine readable and executable instructions can be accessed by the one or more processors 302. The machine readable and executable instructions may comprise logic or algorithm(s) written in any programming language of any generation (e.g., 1GL, 2GL, 3GL, 4GL, or 5GL) such as, for example, machine language that may be directly executed by the processor, or assembly language, object-oriented programming (OOP), scripting languages, microcode, etc., that may be compiled or assembled into machine readable and executable instructions and stored on the one or more memory modules 306. Alternatively, the machine readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components. The memory modules 306 are discussed in more detail below in connection with FIG. 4.

Referring still to FIG. 3, the example ECU 250 includes the data storage component 308. The data storage component 308 may store data captured by the hematology analyzer 102 (FIG. 1), as disclosed in further detail below. The data storage component 308 may also store other data used by the various components of the ECU 250.

In embodiments, the ECU 250 comprises network interface hardware 310 for communicatively coupling the hematology analyzer 102 (FIG. 1) to the morphology analyzer 104 (FIG. 1) and/or the user interface device 106 (FIG. 1). This may allow data to be shared between the devices to improve the data collected by the devices, as disclosed herein.

In some embodiments, the ECU 250 comprises the output device 312. The output device 312 can include a graphical user interface (GUI), a screen, one or more devices in communication with the one or more processors 302 (such as smartphones, tables, and the like), and/or any other device or interface suitable for displaying data. In some examples, the output device 312, or another device, may be configured to as an input device to receive user input.

Referring to FIGS. 3 and 4, the one or more memory modules 306 include a sensor data reception module 400, a cell classification module 402, a cell parameter determination module 404, a confidence level determination module 406, a cell complexity determination module 408, a corrected parameter reception module 410, and an algorithm update module 412. Each of the sensor data reception module 400, the cell classification module 402, the cell parameter determination module 404, the confidence level determination module 406, the cell complexity determination module 408, the corrected parameter reception module 410, and the algorithm update module 412 may be a program module in the form of operating systems, application program modules, and other program modules. Such a program module may include, but is not limited to, routines, subroutines, programs, objects, components, data structures and the like for performing specific tasks or executing specific data types as will be described below.

The sensor data reception module 400 may receive data from the various components of the hematology analyzer 102 (FIG. 1). In particular, the sensor data reception module 400 may receive signals corresponding to sensed/measured energy from the one or more sensors 225, 230, 232, and 235 (FIG. 2). Such signals may be referred to generally as “sensor signals” and may be referred to more specifically as, e.g., SS sensor signal, FSL sensor signal, etc. The sensor data reception module 400 may convert the sensor signals to data (e.g., digital values) indicative of, for examples, amounts of SS, FSL, FSH, EXT, fluorescence, or other energy, sensed/measured by sensors, or indicative of TOF or other metrics. As used herein, data converted from sensor signals will be referred to generally as “sensed data” and may be referred to more specifically as, e.g., sensed SS data, sensed FSL data, etc.

The cell classification module 402 may classify various cells of a blood sample based on the data received by the sensor data reception module 400. In embodiments, the cell classification module 402 may classify the cells of the blood sample using machine-learning algorithms. The cell classification module 402 may classify the identified red blood cells, white blood cells, and platelets into sub-types, using cell sub-type machine-learning algorithms.

In aspects of the disclosure, the machine-learning algorithms of the cell classification module 402 are trained and validated/tested on a large collection of patient sample data. In a training phase, a set of features is extracted from a collection of patient sample data and provided as training data to one or more machine learning models, such as neural networks, as inputs. The machine learning models learn the patterns present in the data they are given and use an error between the expected and actual output to correct themselves by adjusting their parameters as more data is input (for example, by correcting the weights and biases for each connected pair of neurons in a neural network). The expected outcome can be provided by annotated ground truth data associated with each patient sample. In some embodiments, validation and testing of the trained machine learning models is performed to ensure that the models are generalized (they are not overfitted to the training data and can provided similar performance on new data as on the training data).

In some aspects of the present disclosure, a portion of the patient sample data is held back from the training set for validation and testing. The validation dataset is used to estimate the machine-learning model's performance while tuning the model's parameters (such as the weights and biases of a neural network). The test dataset is used to give an unbiased estimate of the performance of the final tuned machine-learning model. It is well known that evaluating the learned model using the training set would result in a biased score as the trained model is, by design, built to learn the biases in the training set. Thus, to evaluate the performance of a trained machine-learning model, one needs to use data that has not been used for training.

In some aspects of the disclosure, the collected patient sample data set can be divided equally between the training set and the testing set. The machine learning models are trained using the training set and their performance is evaluated using the testing set. The best performing machine-learning model may be selected for use. The machine-learning model is considered to be generalized or well-trained if its performance on the testing set is within a desired range of the performance on the training set. If the performance on the test set is worse than the training set (the difference in error between the training set and the testing set is greater than a predefined threshold), a two-stage validation and testing approach may be used.

In some aspects of the disclosure, in a two-stage validation and testing approach, the collected patient sample data set is divided between the training set, the validation set, and the testing set. The machine learning models are first trained using the training set, then their parameters are adjusted to improve their generalization using the validation set, and, finally, the trained machine learning models are tested using the testing set. The patient sample data set may be divided equally between the desired training, validation, and testing sets. This works well when there is a large collection of data to draw from. In cases where the collection of data samples is limited, other well-known techniques, such as leave one out cross validation and testing or k-fold cross validation may be used to perform validation and testing. Cross-validation is a resampling procedure used to evaluate machine-learning models on a limited data set. The procedure has a single parameter called k that refers to the number of groups that a given data set is to be split into. As such, the procedure is often called k-fold cross-validation. When a specific value for k is chosen, such as k=10, the procedure becomes 10-fold cross-validation.

Cross-validation is primarily used to estimate how the trained model is expected to perform in general when used to make predictions on data not used during the training of the model. The dataset is shuffled randomly and divided into a predefined number (k) of groups. The training and testing process is performed k times, with one of the groups of data being held out as the testing set for each iteration and the remaining k−1 groups being used as the training set. Each model is fitted (trained) on the training set and evaluated (tested) on the test set to determine the level of generalization of the trained models.

In addition to preventing overfitting, k-fold cross validation can also help determine the model structure and the parameter training process for the machine-learning model. For example, a neural network model can have one or more “hidden” layers of neurons between the input layer and the output layer. Further, different neural network models can be built with different numbers of neurons in the hidden layers and the output layers. In some aspects of the disclosure, in the training phase, a plurality of machine-learning models (for example, neural network models having different numbers of layers and different numbers of neurons in each layer) are generated. Each of the plurality of machine learning models is trained using k-fold cross validation, resulting in a score that predicts the skill of each model in providing the correct expected output. The model (for example, number of layers and number of neurons in each layer of a neural network) having the highest predictive score is selected and then trained (or further trained), using a larger portion of the patient sample data to generate the final machine learning model, which may be trained and/or retrained over time based on updated analysis and data across the patient sample data, as well as new and/or updated patient data that may be used to further refine and/or train the model. Other examples are possible.

In some aspects of the disclosure, the machine-learning model is a convolution neural network. Inspired by the human visual system, CNNs utilize convolutional layers to extract local patterns and hierarchical representations from the input data. This ability to automatically learn and recognize intricate features makes CNNs particularly suitable for medical data classification tasks.

The architecture of a CNN comprises several layers, each serving a specific purpose in the classification process. The primary layers in a typical CNN architecture for medical diagnostics are convolution layers, pooling layers, activation functions, and fully connected layers. Convolutional layers perform convolution operations using learnable filters, detecting local patterns and features in the medical data. By capturing information at multiple scales, CNNs can identify important structures and abnormalities. Pooling layers reduce the spatial dimensions of the feature maps obtained from convolutional layers. Common pooling techniques, such as max pooling, down sample the feature maps while retaining the most salient information. This spatial reduction helps reduce computational complexity and enhances translation invariance. Activation functions introduce non-linearity to the network, enabling CNNs to model complex relationships within the medical data. Popular activation functions include ReLU (Rectified Linear Unit) and sigmoid, which enhance the network's ability to learn discriminative features. Fully connected layers connect all neurons from the previous layer to every neuron in the subsequent layer. These layers integrate the learned features and make the final classification predictions. In medical diagnostics, the output layer typically represents the different disease classes or diagnostic outcomes.

The training of CNNs involves two key processes: forward propagation and backpropagation. During forward propagation, the input medical data passes through the layers of the network, and the predictions are generated. These predictions are then compared with the ground truth labels to calculate the loss. Backpropagation involves calculating the gradients of the loss function with respect to the network's parameters and adjusting those parameters using optimization algorithms such as stochastic gradient descent (SGD) or Adam. The process of forward propagation and backpropagation is iteratively repeated on a training dataset until the network learns to accurately classify medical data.

Machine learning models according to the present disclosure are not limited to neural networks, and any suitable other or combination of other machine learning models, such as a Markov random field network, support vector machine, random forest of decision trees, or k-nearest neighbor, or the like may be used to provide diagnostic and/or treatment information from patient sample data.

In some aspects of the disclosure, the cell classification module 402 is refined over time, using continuous learning techniques, as new patient sample data is collected at the hematology analyzer 102 (FIG. 1), and the morphology analyzer 104 (FIG. 1) as described in greater detail herein. Continuous learning is the ability of a trained machine-learning model to autonomously learn and adapt in field use (production) as new data comes in. Continuous learning mimics the human ability to continually acquire, fine-tune, and transfer knowledge and skill throughout a person's lifespan.

Although continuous learning machine-learning models may sound ideal for medical purposes, in practice, there are many long-standing challenges in applying them. One main obstacle with continuous learning is catastrophic forgetting (or catastrophic interference phenomenon), in which the new information interferes with what the machine-learning model has already learned. This can lead to an abrupt decrease in performance while the new data is being integrated, or even worse, an overwrite of the model's previous knowledge with the new data. Most of the current applications for continuous learning in nonmedical fields are less critically impacted by this limitation. However, the stakes for real-time medical applications of machine learning are high due to their impact on health outcomes.

A simple solution to catastrophic forgetting is to completely retrain the model each time new data is available, but this can be computationally expensive and inhibit real-time inferences. While advances in cloud computing may provide a solution to this problem of computational complexity and cost, the GPU accelerated resources that are needed to retrain on the full datasets are complex to create and are difficult to securely maintain. Moreover, healthcare information governance across different countries is constantly evolving, making it difficult to maintain compliance. In addition, the availability of retrospective training sets needed to fully retrain the model with new data is especially challenging in healthcare due to consent for use constraints. Thus, completely retraining the trained machine learning models on both the old data and the new data may not be feasible.

Furthermore, in the United States, only a few automated algorithms have been approved by the Food and Drug Administration (FDA) for limited capacities such as detection of diabetic retinopathy or breast abnormalities. All of these algorithms have been “locked” for safety, to prevent any potential for further learning or change post-approval. However, continual learning (i.e., “unlocked”) ML models may be more advantageous as they are able to incrementally learn from their mistakes and fine-tune their performance with progressively more data, similar to the ways that human clinicians learn.

There are certain areas within clinical medicine where continual learning ML models could be safely implemented. One example is diagnostic testing, but the labeling of the new data would be a rate-limiting step. When new patient data becomes available, the trained model would perform inference and make a diagnostic prediction. The new data would also need to be manually graded using the reference standard, and the results would then be used to update the model. Manual image grading is a time-consuming step that will limit the overall utility of an automated AI algorithm since all new incremental data will require human input to produce reliable labels, but the performance of the model as it “learns” would not directly affect patient outcomes.

Various techniques, such as regularization, rehearsal, dynamic architecture, memory-augment models, and generative replay, may be used to prevent catastrophic forgetting in continual learning.

In some aspects of the disclosure, regularization techniques can be used to prevent overfitting of the trained ML model to the new data. Overfitting occurs when the model becomes too specialized on the new data and forgets the previously learned knowledge. Regularization techniques can be used to penalize complex models that are more likely to overfit. The most commonly used regularization techniques are weight decay, dropout, and early stopping. In the context of a neural network type ML model, weight decay involves adding a penalty term to the function that penalizes large weights. This technique encourages the model to use small weights, which can help prevent overfitting. Dropout is another regularization technique that randomly drops out some of the neurons in a neural network model during training. This technique can help prevent the model from becoming too specialized on the new data. Early stopping is another commonly used regularization technique that stops the training of the model when the performance on the validation set stops improving. This technique prevents the model from overfitting to the new data.

In some aspects of the disclosure, in rehearsal techniques, the trained ML model is retrained on the new data along with some previously training data to prevent forgetting. This can be achieved by storing some of the previous training data and randomly selecting some of it to be used during training on the new data. Rehearsal can be done using several strategies such as random selection, prioritized selection, or intelligent selection. Random selection involves randomly selecting some of the previous training data during retraining with new data. Prioritized selection involves selecting the most important previous data based on some criteria. Intelligent selection involves selecting the previous data that is most relevant to the new data.

In some aspects of the disclosure, the third technique used to prevent catastrophic forgetting is dynamic architecture. Dynamic architecture refers to modifying the architecture of the trained ML model based on the new data to prevent catastrophic forgetting. This can be done by, for example, adding or removing neurons or layers in a neural network ML model based on the new data. The idea is to allow the model to adapt to the new data while preserving the previously learned knowledge. However, modifying the architecture of the model can be computationally expensive and requires careful tuning.

In some aspects of the disclosure, memory-augmented networks are used to incorporate external memory modules that allow the trained ML model to store and retrieve information. This approach can help prevent forgetting by allowing the ML model to explicitly store information about the previously learned tasks. Memory-augmented networks can be divided into two categories: ML models with external memory and ML with internal memory. ML models with external memory include models like Neural Turing Machines (NTMs), Differentiable Neural Computers (DNCs), and Memory Networks (MNs). ML models with internal memory include models like Long Short-Term Memory (LSTM), Gated Recurrent Units (GRUs), and Transformer-based models.

Similar to rehearsal techniques, a fifth technique to prevent catastrophic forgetting, in some aspects of the disclosure, is generative replay. Generative replay involves generating synthetic samples of the previous training data and using them to train the ML model on the new data. This approach has been shown to be effective in preventing forgetting and can be combined with other techniques for better performance. Generative replay can be done using several strategies such as generative adversarial networks (GANs), variational autoencoders (VAEs), or mixture density networks (MDNs). The idea is to generate synthetic samples that are similar to the previous training data and use them to train the model on the new data.

In some aspects of the disclosure, the cell classification module 402 is refined over time, using continuous learning techniques, as new patient sample data collected at the hematology analyzer 102 (FIG. 1) is associated with data from the morphology analyzer 104 (FIG. 1), as described in greater detail herein.

The cell parameter determination module 404 may determine parameters associated with the cell types identified by the cell classification module 402. The cell parameter determination module 404 may utilize cell parameter machine-learning algorithms to determine parameters of cell types.

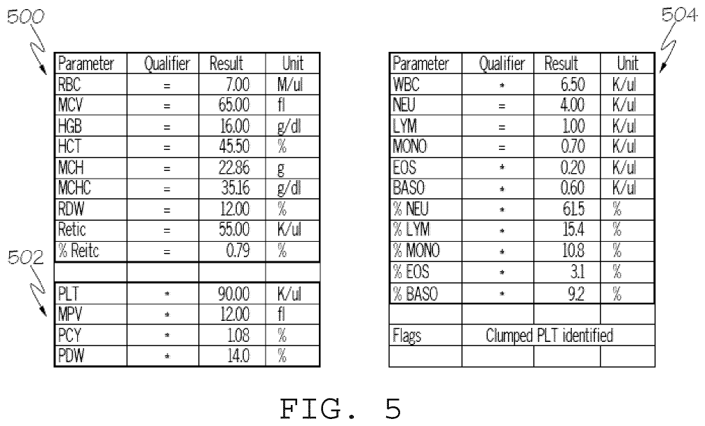

FIG. 5 shows a table of example parameters that may be determined by the cell parameter determination module 404. In the example of FIG. 5, section 500 shows parameters associated with red blood cells, section 502 shows parameters associated with platelets, and section 504 shows parameters associated with white blood cells.

In the example of FIG. 5, the parameters associated with red blood cells that may be determined by the cell parameter determination module 404 (FIG. 4) include total red blood count (RBC), mean corpuscular volume (MCV), hemoglobin (HGB), hematocrit (HCT), mean corpuscular hemoglobin (MCH), mean corpuscular hemoglobin concentration (MCHC), red distribution width (RDW), reticulocyte count (Retic), and percentage of reticulocyte (% Retic). In the example of FIG. 5, the parameters associated with platelets that may be determined by the cell parameter determination module 404 (FIG. 4) include platelet count (PLT), mean platelet volume (MPV), plateletcrit (PCT), and platelet distribution width (PDW). In the example of FIG. 5, the parameters associated with white blood cells that may be determined by the cell parameter determination module 404 include white blood count (WBC), absolute neutrophil count (NEU), absolute lymphocyte count (LYM), absolute monocyte count (MONO), absolute eosinophil count (EOS), absolute basophil count (BASO), percentage neutrophils (% NEU), percentage lymphocytes (% LYM), percentage monocytes (% MONO), percentage absolute eosinophils (% EOS), and percentage basophils (% BASO). However, it should be understood that in some examples, the cell parameter determination module 404 may determine other parameters associated with the blood sample.

Referring to FIGS. 4 and 5, in embodiments, the confidence level determination module 406 determines confidence levels associated with one or more of the parameters determined by the cell parameter determination module 404. In particular, the confidence level determination module 406 may determine whether one or more of the algorithms used by the cell parameter determination module 404 are unable to determine one or more of the determined parameters with a confidence level above a predetermined or configurable threshold.

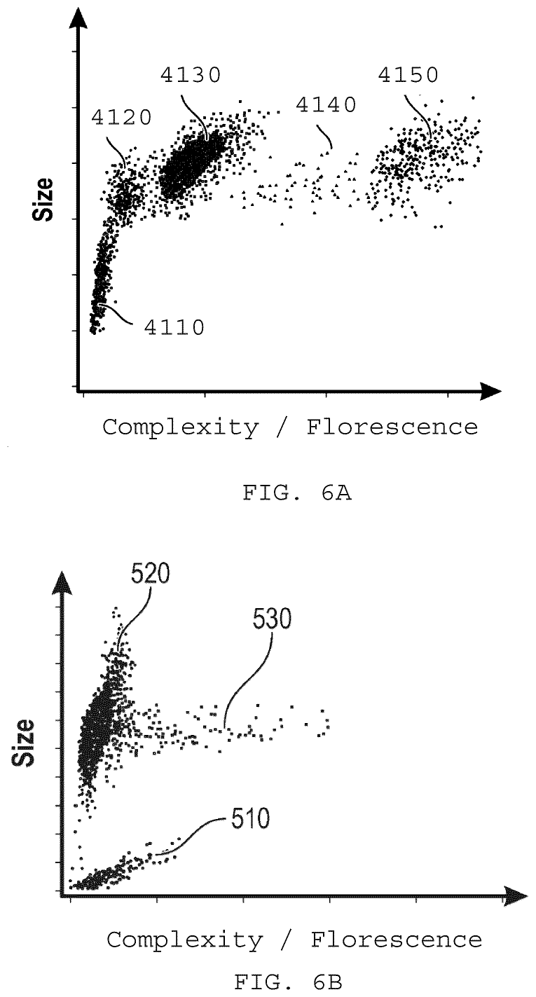

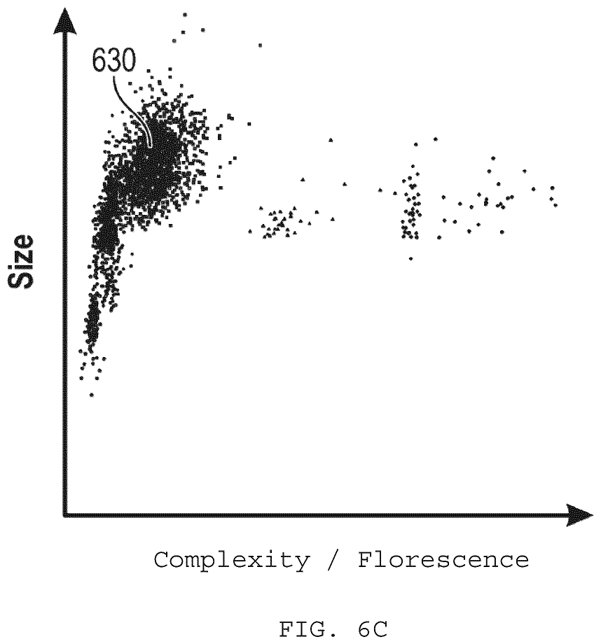

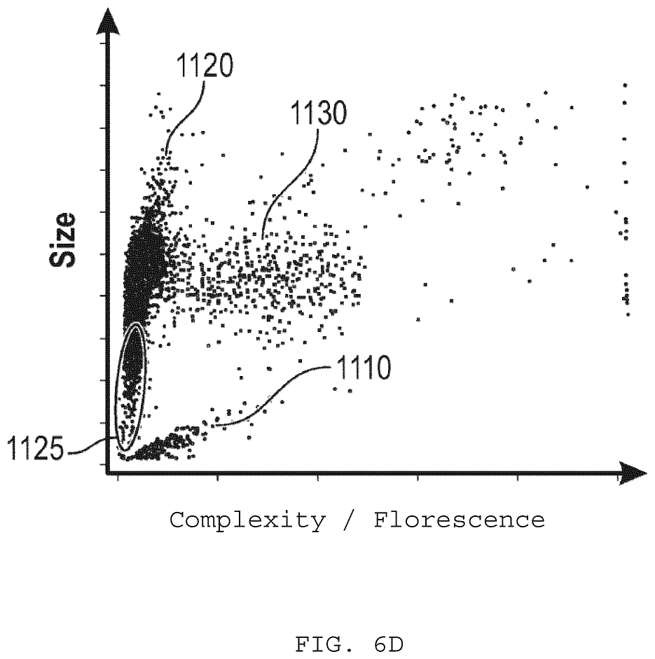

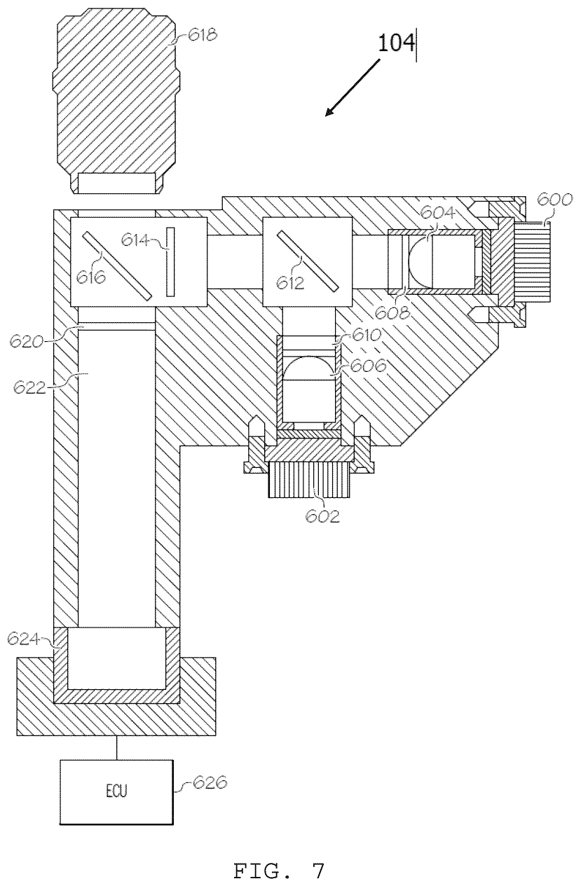

Referring to FIGS. 6A and 6B, in some embodiments, the hematology analyzer 102 outputs graphical representations of cellular features in the form of 1-dimensional histograms and/or 2-dimensional dot plots. In embodiments, the illustrated 2D dot plots have one axis that represents values of complexity and/or florescence of the constituents in a sample and another axis that represents values of size of the constituents in a sample.

In embodiments, the complexity axis of the 2D dot plot corresponds to sensed data of one or more sensors in the hematology analyzer 102 or corresponds to a complexity metric that indicates the complexity of the constituent cells in a hematology sample (e.g., cell shape, degree of development of the nucleus, granules, RNA/DNA, of the constituent cells, etc.). In some embodiments, the complexity is a quantity that is derived from sensed data. For example, the complexity may be a quantity that is computed as a function of sensed SS data, sensed FSL data, sensed FSH data, sensed EXT data, sensed TOF data, sensed fluorescence data, and/or other sensed data. Persons skilled in the art will understand complexity and how to compute complexity.

In embodiments, size is represented by one axis of the 2D dot plot and is a quantity that is derived from the sensed data and/or metrics of the hematology analyzer 102. Persons skilled in the art will understand how to indicate size of constituents using sensed data and/or metrics. For example, the size of cells may be determined based on FSL and/or EXT data.

Without being bound by theory, EXT and FSL sensor signals both have strong sensitivity to size of constituents in a hematology sample, and either signal can be used to indicate size of such constituents. In embodiments, the size of particular constituents (e.g., red blood cell, platelet, etc.) may be indicated using either the EXT sensor signal or the FSL sensor signal. In embodiments, the size of particular constituents may be indicated by considering both the EXT and the FSL sensor signals. The EXT and FSL sensor signals are merely examples, and other sensed data and/or metrics may be used to indicate size. Persons skilled in the art will understand how to derive size of a constituent from image data. For example, the geometric extents of a cell may be identified and size may be determine based on known magnification and pixel resolution, impact of reagents on spherical nature of cells, and/or other factors.

With continuing reference to FIG. 6A, the illustrated 2D dot plot presents white blood cells processed by the hematology analyzer 102. As persons skilled in the art will understand, white blood cells can include various types of cells, including lymphocytes, monocytes, neutrophils, basophils, and eosinophils, and these types of cells may have different sizes and complexities. In this configuration, larger cells will generally appear higher on the size axis, and cells with more complexity (e.g., more irregular shapes, further developed nucleus, granules, RNA/DNA, etc.) will generally appear farther right on the complexity axis. When healthy white blood cells are plotted in a 2D dot plot, the different types of white blood cells may present themselves relative to each other as shown in FIG. 6A.

In the example depicted in FIG. 6A, the first group of dots 4110 are lymphocytes, the second group of dots 4120 are monocytes, the third group of dots 4130 are neutrophils, the fourth group of dots 4140 are basophils, and the fifth group of dots 4150 are eosinophils. An example of a system that can generate the type of 2D dot plot shown in FIG. 6A are the IDEXX ProCyte One and the IDEXX ProCyte Dx hematology analyzers. The 2D dot plot of FIG. 6A and the particular size and complexity described above are merely examples, and other 2D plots with different values indicative of healthy cells are contemplated. As one example and without being bound by theory, different species have different size and complexity values for healthy blood cells. In particular, the 2D plot shown in FIG. 6A is of a healthy canine subject, while the lymphocytes, monocytes, neutrophils, basophils, and eosinophils of a healthy feline subject may have different sizes and complexities along the size axis and the complexity axis.

Referring now to FIG. 6B, the illustrated 2D dot plot presents red blood cells processed by a hematology system. As persons skilled in the art will understand, platelets and reticulocytes appear with red blood cells. Platelets are a component of blood that play a critical role in normal and abnormal hemostasis, and reticulocytes are immature red blood cells. Platelets, reticulocytes, and red blood cells may have different size, complexity, and/or fluorescence. In FIG. 6B, size is represented by one axis of the 2D dot plot, and the separate axis may be indicative of complexity or fluorescence. When healthy red blood cells, platelets, and reticulocytes are plotted in such a 2D dot plot, the different cells may present themselves relative to each other as shown in FIG. 6B. The first group of dots 510 are platelets, the second group of dots 520 are red blood cells, and the third group of dots 530 are reticulocytes. In the illustrated embodiment, the group of dots 520 corresponding to red blood cells does not include red blood cell fragments, such as fragments resulting from lysed red blood cells. The 2D dot plot of FIG. 6B and the size, complexity, and florescence described above are merely examples. For example, and without being bound by theory, different species may have different sizes, complexity, and fluorescence responses for healthy blood samples. In particular, the 2D dot plot shown in FIG. 6B is of a healthy canine, while a blood sample from a healthy feline would have a different size, complexity, and fluorescence values. Further, red blood cells, platelets, and reticulocytes can be plotted in a 2D dot plot having a size axis and another metric or sensed data for the separate axis, as opposed to fluorescence or complexity for the separate axis. The metric or sensed data represented by the separate axis does not need to be (but may be) orthogonal to the size represented by the size axis. Such and other embodiments are contemplated to be within the scope of the present disclosure.

The assignment of a constituent type to a constituent (e.g., a cell) does not mean and is not intended to mean that the assigned type for each detected cell is correct without error. Rather, as mentioned above, the assignment of a constituent type may be performed using heuristic rules, algorithms, and/or machine learning techniques, among other approaches, which have some error rate. A sufficiently low error rate, however, will provide confidence in the assigned constituent types. Examples of systems which assign constituent types to the constituents of a sample are the IDEXX ProCyte Dx hematology analyzer and the IDEXX ProCyte One hematology analyzer.

With reference to FIGS. 6A and 6B, the hematology analyzer 102 generates the 2D dot plots based on the sensed data. The 2D dot plots generated by different systems may be non-identical even when they analyze the same thing (i.e., the same blood sample). This may be caused by, for example, small variations in each hematology system. For example, in embodiments in which the hematology system 102 is a flow cytometer, variations related to optical path differences within the laser module (e.g., due to slight alignment variations or imperfections in optics) and/or slight fluidic variations, among other reasons, can cause variations between hematology systems 102. These variations may be characterized and accounted for using quality control procedures.

In accordance with aspects of the present disclosure, and with reference also to FIGS. 1-4, the hematology analyzer 102 may utilize one or more QC materials that have known properties, e.g., known morphology, size, interactivity with energy, and/or the like. Accordingly, as the QC materials are interrogated by the hematology system (e.g., via flow cytometry or imaging) the QC materials should present on a 2D plot in a known manner. The hematology analyzer 102 may store reference values of sensed data for the QC material(s), may store a reference 2D dot plot that shows known locations of dots for the QC material(s), or may store other reference information relating to the known properties of the QC material(s). At various intervals, the hematology analyzer 102 may interrogate QC material(s) (e.g., via flowing the QC material(s) through the flow cell 215). Information generated from this interrogation can be compared to the reference information to determine adjustments that can normalize the sensed data and/or the dots in a 2D dot plot to match the reference information.

In some embodiments, subtle differences related to the sample path in the hematology system 100 may also affect 2D dot plots, and such differences may not be captured by the QC materials. Rather, quality control that accounts for such variations may be performed based on the cells present in the sample. Adjustments may be made on a sample-by-sample basis to account for variables for that specific sample and to normalize the 2D dot plot. An example of such quality control is described in U.S. Patent Application Publication No. US20150025808A1, which is hereby incorporated by reference herein in its entirety.

In embodiments, the adjustments described above may be computed by the hematology analyzer 102, and the hematology analyzer 102 may apply the adjustments to sensed data and/or to the dots in 2D dot plot for a patient sample to normalize the 2D dot plot. Normalizing the 2D dot plot to account for differences between hematology systems allows the various analyses to not be influenced by instrument-specific factors. The normalization measures described above are merely examples. Other normalization measures are contemplated to be within the scope of the present disclosure, including various measures described in U.S. Pat. No. 11,441,997, which is hereby incorporated by reference herein in its entirety.

In some instances, the analyzer outputs flags associated with certain features or parameters, which can indicate that the analyzer had a fault during the analysis (e.g., a fault indication that the analyzer is running out of reagents or sample), or that there is a quantitative or morphologic abnormality in the sample that requires additional analysis.

Hematology analyzers based on impedance and/or flow cytometry technology tend to have excellent precision and accuracy in quantitative reporting for blood samples. This is due to the strong statistical sampling that is performed on the sample, in which thousands to hundreds of thousands of cells are characterized. This statistical sampling is in stark contrast to microscopy-based manual blood counts that tend to incorporate merely 100 to 400 cells. Furthermore, automated dilution and pipetting systems generally yield precision values that are in the low single digits for percentage cell volume calculations.

However, in situations in which morphologic abnormalities are present, cell populations can no longer be adequately separated and reported in impedance and/or flow cytometry-based systems. For example, morphologic abnormalities may cause certain cell types/populations to present in abnormal ways such that the cells cannot be adequately distinguished from other cell types/populations.

Without being bound by theory, morphological abnormalities can be present in veterinary samples as much as 30% of the time and may be indicative of conditions that warrant additional testing. When the analyzer outputs one or more flags indicative of a morphological abnormality, a blood film may be created for microscopic analysis. Trained professionals, such as pathologists review the blood film for morphologic features of the cells of the sample. This information may be integrated with the rest of the clinical information associated with the sample to derive a diagnosis and a potential treatment plan. However, there are many factors associated with the morphological abnormalities that are often overlooked and/or inconsistently diagnosed by any given clinician or pathologist, much less from clinician to clinician and/or pathologist to pathologist. As such, many patients affected with these morphologic abnormalities will be misdiagnosed and mistreated (or untreated) based on existing methods, which often lead to inconsistent and erroneous hematology analysis and associated treatment plans. Further, as described in more detail below, in addition to more accurately and efficiently diagnosing these morphological abnormalities, the embodiments detailed herein also improve the identification and implementation of treatments that are best suited to treat one or more specific morphological abnormalities, which otherwise would not (and could not exist) without the technological improvements described herein.

As one example, left shift is a morphological abnormality that is difficult to identify. Without being bound by theory, one indication of inflammation is that white blood cell populations in a blood sample contain a higher proportion of immature cells. For example, white blood cell populations in a blood sample from a subject with inflammation may have a higher proportion of immature neutrophils, which occurs as inflammatory cytokines stimulate bone marrow to produce neutrophils and release mature and immature neutrophils into the blood. Toxic change in neutrophils is another finding that is associated with inflammation.

Referring to FIG. 6C, in blood samples indicating inflammation, the neutrophils 630 tend to be larger and less complex than normal neutrophils, so in the 2D dot plot, the dots for the neutrophils 630 tend to shift up and to the left along the depicted axes, which is referred to herein as “left shift.” This can be seen in comparing, for example, the spatial distribution of neutrophil dots 430 of FIG. 6A (no inflammation) with the spatial distribution of neutrophil dots 630 of FIG. 6C (inflammation).

While the upward and leftward shift of the dots in FIG. 6C is pronounced for illustration as compared to the healthy sample depicted in FIG. 6A, in many instances, the identification of left shift is difficult. In particular, many physicians and veterinarians are not trained in interpreting dot plots. Moreover, for a variety of reasons, including the variability between samples and hematology analyzers, and that the difference in the presentation of healthy cells (FIG. 6A) and cells indicating inflammation (FIG. 6C) is one of degree, it is difficult to determine clear objective thresholds for identifying cells exhibiting left shift.

Instead, indications of inflammation as described above can generally be identified by manual human analysis of blood films under a microscope, but not always. For example, a skilled pathologist can identify and quantify immature neutrophils and toxic neutrophils, but such skilled pathologists are not always available for patients and, even when available, often produce inconsistent diagnoses and/or treatment plans between them. Further, immature forms of neutrophils may be manually identified by their maturation stage using blood films. The maturation stages from most to least mature are as follows: mature segmented neutrophils, bands, metamyelocytes, myelocytes, promyelocytes and myeloblasts. When inflammation occurs, less mature forms can be present in the blood films. Inflammation also produces toxic change in neutrophils in the form of morphologic changes in the cytoplasm (e.g., increased basophilia, vacuolation, granulation, Dohle bodies) and can result in the presence of larger neutrophils if nuclear divisions are skipped.

Without being bound by theory, accurate identification of left shift and/or toxic change is an important step for veterinarians to diagnose and treat non-human animals. As noted above, left shift and/or toxic change are indications of the presence of inflammation. Inflammation can result from a variety of maladies, for example and without limitation, infectious agents (e.g., bacteria, viruses, protozoa, fungi, etc.), auto-immune disease, neoplasia, necrosis, and/or the presence of a foreign body.

Without the identification of left shift and/or toxic change, inflammation can be difficult to detect in non-human animals. Symptoms of inflammation can be non-specific, including lethargy, decreased appetite, vomiting, diarrhea, and the like. Moreover, inflammation can be internal, such that there are no external physical signs of inflammation. Further, symptoms of inflammation may not be consistent between different non-human animals with the same underlying condition. Further still, as readily understood by the reader, non-human animals are incapable of speech and cannot describe their symptoms or conditions. As such, the identification of left shift and/or toxic change is important to the appropriate diagnosis of inflammation in non-human animals, and failing to appropriately identify left shift and/or toxic change may lead to missed diagnosis and suboptimal health outcomes.

Accurately recognizing immature neutrophils and toxic change on blood films requires significant training, and even then, accurate identification and quantification via a human review of a blood film can still be plagued by individual subjectivity. In addition, immature neutrophils and toxic change can sometimes be evident in only a small subset of neutrophils present in the sample and may, therefore, be easy to miss in the blood film if a cursory evaluation is performed.

Moreover, in many circumstances, blood films are not typically prepared as part of a blood analysis. Instead, many practitioners rely on results from a hematology system to provide an initial analysis before proceeding to prepare a blood film. However, many practitioners are not familiar with 2D dot plots and may have difficulty accurately identifying conditions indicative of inflammation. Consequently, they may fail to prepare blood films for analysis. Moreover, morphologic characteristics seen with immature neutrophils and toxic change occur as a continuum, and artifactual changes that mimic immature neutrophils and toxic change can occur in aged samples. As such, failure to timely prepare and analyze blood films can result in an inability to correctly identify conditions indicative of inflammation, which, if left undiagnosed or untreated, can cause irreversible damage and detriment to the patient.

However, by accurately identifying left shift and/or toxic change, appropriate diagnoses and treatments can be administered. In embodiments, in response to identifying left shift and/or toxic change, a user administers one or more of antibiotics, supportive treatment including one or more of fluids or electrolytes, anti-inflammatory food, immune suppressants, or chemotherapy to the non-human animal.

As another example, small pathologic RBC present another morphologic abnormality that is difficult to detect using a hematology analyzer. Small-pathologic red blood cells are typically identified after examining a blood film. Due to the various mechanisms that produce SP-RBC, these cells have different morphologies that can guide identification of the underlying pathologic process, but are often left undetected by clinicians. Thus, importantly, consistent and accurate identification of distinct red blood cell morphology changes can indicate underlying nonspecific disease or lead directly to identification of the specific primary pathologic process.

As an example, immune-mediated hemolytic anemia (IMHA) is a condition where anemia results from immune-mediated destruction of red blood cells. During this process, in the majority of cases, antibody coats the red cells, which signals macrophages to remove a portion of the red cell membrane. As macrophages extract pieces of the membrane, spherocytes (smaller appearing red cells with decreased central pallor) are produced. Initially, these spherocytes are similar in size to normal red blood cells since primarily cell membrane is lost and overall cell volume remains normal; however, as these cells interact with the macrophages, greater and greater amounts of cytoplasm is lost and the overall red blood cell size decreases. Spherocytes are a key diagnostic feature of IMHA and have been reported to occur in up to 90% of dogs with IMHA. Identifying many spherocytes can lead the clinician to make critical therapeutic decisions for treating the anemic patient—but this identification must be accomplished in more consistent and timely manner than clinicians are able to accomplish without the improvement and embodiments described herein.

Like inflammation, symptoms of IMHA are non-specific and IMHA is difficult to diagnose without identifying small pathologic RBC. With appropriate treatment, IMHA can often be managed. In embodiments, in response to identifying IMHA via identifying small pathologic RBC a user may administer one or more of a blood transfusion, fluid therapy, immunosuppressive medication, antibiotics, anticoagulant medication, gastrointestinal medication configured to restrict stomach bleeding or ulcers, or any combination thereof.

As another example, oxidative injury to red blood cells results from exposure to some drugs (e.g., acetaminophen), oxidative agents (onions, zinc), and in association with certain disease processes (e.g., neoplasia, diabetes). Oxidative injury can denature hemoglobin which produces Heinz bodies, or damage red cell membranes, generating eccentrocytes, blister cells and keratocytes. All mechanisms result in smaller than normal erythrocytes. When oxidative injury is marked, it can result in secondary hemolytic anemia. If the anemia is primarily the result of oxidative damage, identification and removal of the inciting cause is crucial for treatment and, if left undiagnosed or untreated, can cause irreversible damage and detriment to the patient.

In embodiments, in response to identifying small pathologic RBC associated with oxidative stress, a user may administer to a non-human animal, an antioxidant-rich diet, ascorbic acid, resveratrol, N-acetylcysteine, omega-3 fatty acid supplements, or any suitable combination thereof.

With regard to metabolic and membrane disorders, there are nonspecific red blood cell changes that can occur secondary to alternations or injury of the red cell membranes. Although the changes are nonspecific, they can indicate underlying disease that could otherwise be undetected. Certain morphologies can suggest a selected list of more common differentials that can aid the clinician's diagnostic choices. Blister cells/keratocytes occur after alterations or injury to the red blood cell membrane and can be associated with different underlying causes (e.g., iron deficiency, oxidative injury, liver disease, microangiopathic disease). Acanthocytes are thought to be produced by alterations in the lipid composition of the red cell membranes or mechanical fragmentation. They are an important indicator of underlying disease and in canines have been associated with a number of processes (e.g., cancer, liver disease, iron deficiency and disseminated intravascular coagulation (DIC)). Lastly, poikilocytosis in feline patients can signal metabolic disease (e.g., liver disease, renal disease, hyperthyroidism) and should prompt further diagnostics when present in significant numbers.

In embodiments, in response to identifying small pathologic RBC associated with metabolic and membrane disorders, a user may administer to a non-human animal iron supplements, an antioxidant-rich diet, ascorbic acid, resveratrol, N-acetylcysteine, omega-3 fatty acid supplements, plasma exchange, blood transfusion, immunosuppressive therapy, thromboprophylaxis, antibiotics, or any suitable combination thereof.

With regard to mechanical injury, schistocytes are red cell fragments and they reflect mechanical injury to red cells. They often form when fibrin strands are present within the microvasculature or when vascular disease results in an abnormal endothelial lining or turbulent blood flow. Some examples of conditions in which schistocytes occur are DIC, vasculitis and hemangiosarcoma. As schistocytes result from fragmentation, they can also occur when other pathologic processes result in the production of red cells with increased mechanical fragility (e.g., secondary to iron deficiency, alternations in red cell membranes).

In embodiments, in response to identifying small pathologic RBC associated with vasculitis, a user may administer to a non-human animal anti-inflammatory medication, immunosuppressive medication, antibiotics, topical treatments, a vitamin-rich diet, an elimination diet, or any suitable combination thereof. In embodiments, in response to identifying small pathologic RBC associated with hemangiosarcoma, a user may perform surgery to remove a tumor or administer chemotherapy.

Iron deficiency can occur because of an iron-deficient diet. However, in canine and feline patients, most cases of iron deficiency result from chronic external blood loss (e.g., gastrointestinal, urinary hemorrhage, parasites). Decreased iron availability will affect erythroid production resulting in smaller cells (microcytes) and cells with reduced hemoglobin concentration (hypochromic cells). Microcytic and hypochromic erythrocytes are key indicators for iron deficiency and cue clinicians to search for underlying causes of blood loss. Capturing concurrent red cell morphology changes significantly aids specificity. After determining a patient has iron deficiency anemia, appropriately chosen diagnostics can expose the primary disease that is resulting in chronic external blood loss. (e.g., neoplasia, ulcers, parasitism)

Because small-pathologic red blood cells are typically identified after examining a blood film, SP-RBC has not been diagnosed by point-of-care hematology analyzers. As described above, pathologic changes in red blood cells can result from a variety of causes, and many of the mechanisms ultimately result in the generation of smaller red blood cells that have decreased cell volume. Different species, such as cats and dogs, will have different size red blood cells, but they generally show a clear distribution of red blood cells exhibiting SP-RBC.

For example, FIG. 6D shows a 2D dot plot of platelets 1110, red blood cells 1120, reticulocytes 1130, in a patient that has SP-RBC. When SP-RBC are present, they form a specific population 1125 that falls below the natural, mature RBC population and stretches towards the platelet population 1110. Compare, for example, the red blood cell dots 520 of FIG. 6B (no SP-RBC), which are not stretched towards the platelet population 510. Further, this population of red blood cell dots 1125 that stretches towards the platelet population 1110 is distinct from red blood cell fragments that may have lysed during the sample preparing process.

Like the identification of left shift, samples exhibiting SP-RBC are difficult to identify using a hematology analyzer alone. While the difference in population distribution between FIG. 6D (SP-RBC) and 6B (healthy sample) is pronounced for illustration, it should be understood that in practice, the distinction in population distribution may be difficult to discern. Moreover and as noted above, many physicians and veterinarians are not trained in interpreting dot plots. Accordingly, morphologic analysis is often required to identify SP-RBC.

As another example, clumped platelets present another morphologic abnormality that can be difficult to identify. In examples, platelets in a sample may clump together, and the clumped platelets may appear as a single constituent. More particularly, clumped platelets can appear similar in size and structure to white blood cells, leading to an inaccurately low platelet count and/or an inaccurately high white blood cell count. Analyzers (flow cytometers and the like) may accurately identify the presence of clumped platelets in some circumstances, thereby indicating that a blood film should be performed to obtain an accurate platelet and/or white blood cell count.

However, in some circumstances, the analyzer may not provide a flag indicating the presence of clumped platelets. Instead the analyzer may incorrectly indicate that the sample contains a low platelet count and/or a high white cell count. In some settings, blood analysis is performed in preparation for surgery, and a low platelet count may lead medical professionals to delay or cancel planned surgeries, potentially leading to suboptimal health outcomes. By contrast, in response to identifying a true low platelet count (e.g., a low platelet count not associated with clumped platelets), a user may administer steroids, a blood transfusion, or the like to prepare a non-human animal for surgery. Moreover, a high white cell count can be indicative of certain disease states such as cancer, and inaccurate hematology analysis can lead to incorrect diagnoses and/or unnecessary treatment. Thus, in addition to more accurately and efficiently diagnosing these morphological abnormalities, the embodiments detailed herein also improve the identification and implementation of treatments that are best suited to treat one or more specific morphological abnormalities, which, if misdiagnosed, could lead to costly and health-altering treatments (e.g., if a patient was misdiagnosed with cancer).

As yet another example, in some instances reagents are not able to lyse the RBC population, and the un-lysed RBC appear on a white cell dot plot (e.g., FIG. 6B) and cannot be distinguished from lymphocytes. Accordingly, in such situations, lymphocyte and WBC values may be artificially inflated, which can lead to incorrect diagnoses.

While each of these conditions can be identified using a blood film, as described above, results from the hematology analyzer 102 may be ambiguous and may fail to clearly indicate that a blood film should be performed. Moreover, manual preparation and evaluation of a blood film may be difficult. In particular staff may not be sufficiently trained to properly evaluate and interpret the blood film. Moreover, proper blood film preparation is technically challenging and many staff may struggle to properly prepare blood films without damaging the blood sample. An alternative to evaluating a blood film at a veterinary practice is to send a blood tube to a reference lab, where a blood film can be made and read by a clinical pathologist. However, it typically takes several hours to several days to receive the results from such a lab. For example, in veterinary contexts, the reference lab may be located distant from the point-of-care, and it may take several hours to a day or more to receive results. In some instances, a blood film is prepared at the point-of-care, and images of the blood film are sent to a clinical pathologist. However, it may take several hours to several days for the pathologist to review the blood film images. In embodiments disclosed herein, a digital microscope is disclosed that may perform the analysis and interpretation of blood morphology.

In some instances, a hematology analyzer may flag certain parameters to indicate that the analyzer has low confidence in those reported parameters. In these instances, it may be desirable to utilize an alternate method or additional testing to evaluate morphologic abnormalities. In some cases, the populations present in a manner that is indicative of specific morphologic changes and can lend additional information to the microscopy assessment.

Morphologic analyses of whole blood samples typically uses microscopy techniques. These approaches are typically performed manually and do not share the same level of precision and accuracy for counts as an automated hematology analyzer. This can be attributed to a variety of factors including manual pipetting, dilution, and limited sampling statistics under the microscope. In addition, there is typically not a useful method of calibration available, since the method of creating a blood film inherently incorporates variability in the absolute counts of cells as they can be pushed to the feathered edge, for example.

The reduction in precision and accuracy of this morphologic analysis is generally acceptable since the reported values from the manual assessment are usually referenced with respect to other indications on the slide. For example, a manual differential looking for the five cell characterizations that make up WBC (NEU, LYM, MONO, EOS, and BASO) may be presented by evaluating 100 random white blood cells and reporting how many of those 100 white blood cells were NEU, and reporting it as a percentage. Similar analysis may be performed for the other four cell characterizations. If there are additional nucleated cells found in the sample, such as bands, toxic neutrophils, or nucleated RBC, they may also be represented as percentage of the 100 nucleated cells evaluated.

Reference laboratories have existing approaches when predetermined criteria are met for white cell differential or clumped platelet conditions that cause concern with the automatically reported output of an automated hematology analyzer. In these instances, an evaluation of a blood film can provide a corrected differential, based on, for example, the 100-cell evaluation described above, and the automated differential can be updated without changing the total WBC value. Similarly, if clumped PLT are identified in the automated results, there is a risk that the PLT value is incorrect. In this instance, a manual evaluation of PLT in the sample can provide a means to identify whether PLT are adequate.

In instances in which a hematology analyzer (e.g., a flow cytometer) and a morphology analyzer (e.g. a digital microscope) can analyze samples from the same subject, the morphology analyzer can provide information relating to cell morphology that is undetected by the hematology analyzer. Moreover, in instances in which a hematology analyzer and a morphology analyzer analyze samples from the same subject, each analyzer may report different values for the same parameters, and it may be difficult for the user to interpret the conflicting results. Accordingly, disclosed herein are systems and methods for harmonizing outputs from a hematology analyzer and a morphology analyzer. Moreover, disclosed herein are systems and methods for improving the function of a hematology analyzer utilizing the output of a morphology analyzer.

When morphologic abnormalities are present in a blood sample, the cell classification module 402 may not be able to classify cell types with a desired accuracy and/or the cell parameter determination module 404 may not be able to determine cell parameters with a desired accuracy.

As shown in FIG. 5, when the cell parameter determination module 404 is unable to determine one or more cell parameters with a desired accuracy, in some embodiments, the cell parameter determination module 404 marks the associated parameter values with a flag or qualifier. In the example of FIG. 5, the various parameters associated with the example blood sample are presented with a “Qualifier” flag of either “=” or “*”. In the example of FIG. 5, a qualifier of “=” indicates that the cell parameter determination module 404 was able to determine the associated parameter with a confidence level above a predetermined threshold, and a qualifier of “*” indicates that the cell parameter determination module 404 was not able to determine the associated parameter with a confidence level above the predetermined threshold. Furthermore, the example of FIG. 5 indicates that clumped PLT were identified. However, in some examples, the confidence level determination module 406 may output other types of confidence metrics.

In the example of FIG. 5, the parameters flagged with “*” indicate that additional analysis is needed to confirm the accuracy of those parameters. In embodiments disclosed herein, the morphology analyzer 104 is used to perform this task, as disclosed in further detail below.

Referring back to FIG. 4, the cell complexity determination module 408 may output information about the complexity of the cells in the analyzed blood sample. In particular, the cell complexity determination module 408 may output graphical representations of cell complexity in the form of the 1-dimensional histograms and/or 2-dimensional dot plots as shown in FIGS. 6A-6D.

The corrected parameter reception module 410 may receive corrected cell parameters from the user interface device 106, as discussed below. In particular, as discussed below, in some examples, the user interface device 106 may receive the parameters determined by the cell parameter determination module 404 along with data from the morphology analyzer 104 and may correct the parameters determined by the cell parameter determination module 404. In some examples, the corrected parameters may be transmitted by the user interface device 106 to the hematology analyzer 102 and may be received by the corrected parameter reception module 410.

After the corrected parameter reception module 410 receives corrected parameters, the algorithm update module 412 may update the algorithms used by the cell parameter determination module 404 based on the corrected parameters. As discussed above, the cell parameter determination module 404 may utilize various algorithms to determine cell parameters. However, these determined parameters may not be accurate or may be determined with low confidence. As such, by receiving corrected cell parameters, the algorithm update module 412 may refine these algorithms based on the corrected parameters to improve the performance of the cell parameter determination module 404 for future use. For example, the algorithm update module 412 may recognize patterns in the type of parameter data that is typically corrected or may perform supervised learning based on the corrected parameters to improve the performance of the cell parameter determination module 404. In particular, the algorithm update module 412, utilizing data (e.g., corrected parameters) from the morphology analyzer 104 (FIG. 1), can make changes to the cell parameter determination module 404 to better recognize phenomenon such as left shift, SP-RBC, clumped platelets, and the like, as described in greater detail herein.

By updating the cell parameter determination module 404 based at least in part on data from the morphology analyzer 104, a balance between including fresh data and maintaining consistency with historical data to avoid concept drift or model instability can be struck. Moreover, by correcting parameters utilizing data from the morphology analyzer 104, the performance of the cell parameter determination module 404 can be improved. As an example, sample data from the sensor data reception module 400 may be unclear (e.g., cells cannot be accurately categorized at an acceptable confidence level). However, proper cell categorization can be determined utilizing data from the morphology analyzer 104. With the proper cell categorization, the machine-learning algorithm of the cell parameter determination module 404 can be changed to reflect the proper cell categorization associated with the sample data. In this way, the effectiveness of the cell parameter determination module 404 at identifying proper cell categorization can be improved as compared to a machine-learning algorithm that does not have access to data from a morphology analyzer 104, as described in greater detail herein.

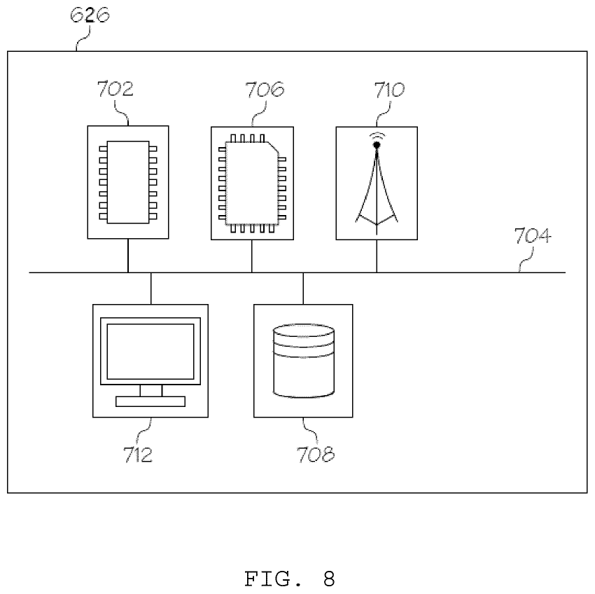

FIG. 7 illustrates a schematic view of the morphology analyzer 104 of FIG. 1. As discussed above, the morphology analyzer 104 is a digital microscope, which includes a fluorescent blue energy source 600, a fluorescent ultraviolet energy source 602, collector lenses 604 and 606, a blue excitation filter 608, an ultraviolet excitation filter 610, an excitation dichroic 612, a field lens 614, an imaging dichroic 616, an objective lens 618, a triband filter 620, a tube lens 622, an imaging sensor 624, and an electronic control unit (ECU) 626. In some examples, the morphology analyzer 104 may include a brightfield source spaced apart from the objective lens 618. For example, in examples in which the morphology analyzer 104 is an inverted digital microscope, the brightfield source is positioned above the objective lens 618. In examples in which the morphology analyzer 104 is an upright microscope, the brightfield source may be positioned below the objective lens 618. In embodiments, the ECU 626 is communicatively coupled to the energy sources 600, 602, the brightfield source, and the imaging sensor 624. In embodiments, the imaging sensor 624 sends images to the ECU, the images indicative of blood samples illuminated by the energy sources 600, 602 and/or the brightfield source.

In embodiments, the imaging sensor 624 and the energy sources 600, 602 are optically coupled to one another such that electromagnetic energy can be passed from the energy sources 600, 602, to a blood sample, and to the imaging sensor 624. In operation, a blood sample is placed along the path of the fluorescent blue energy source 600, the fluorescent ultraviolet energy source 602, and/or the brightfield energy source. In particular, a portion of a blood sample may be analyzed by the hematology analyzer 102 and another portion of the same blood sample may be analyzed by the morphology analyzer 104. One or more of the fluorescent blue energy source 600, the fluorescent ultraviolet energy source 602, or the brightfield source illuminates the sample, and an image of the sample is captured by the imaging sensor 624. In some embodiments, the image captured by the imaging sensor 624 is transmitted to the ECU 626 for automated analysis, as disclosed herein. The morphology analyzer 104 may utilize microscopy techniques to determine attributes associated with cells of the blood sample. In the illustrated example, the attributes identified by the morphology analyzer 104 comprise abnormalities, as disclosed herein. However, in other examples, the morphology analyzer 104 may identify other attributes.

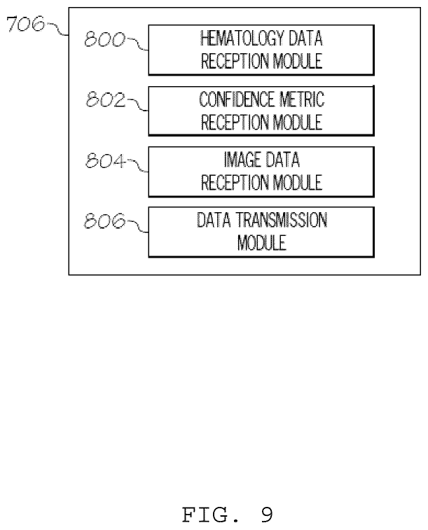

FIG. 8 schematically depicts an example configuration of the ECU 626 of FIG. 7. In the illustrated example, the ECU 626 includes one or more processors 702, a communication path 704, one or more memory modules 706, a data storage component 708, network interface hardware 710, and an output device 712, the details of which will be set forth in the following paragraphs.

Each of the one or more processors 702 may be any device capable of executing machine readable and executable instructions. Accordingly, each of the one or more processors 702 may be a controller, an integrated circuit, a microchip, a computer, or any other physical or cloud-based computing device local to or remote from the morphology analyzer 104 (FIG. 7). The algorithms, including the trained models, signal preprocessing, and noise removal methods discussed below, may be executed by the one or more processors 702. The one or more processors 702 are coupled to a communication path 704 that provides signal interconnectivity between various modules of the ECU 626. Accordingly, the communication path 704 may communicatively couple any number of processors 702 with one another, and allow the modules coupled to the communication path 704 to operate in a distributed computing environment. Specifically, each of the modules may operate as a node that may send and/or receive data.

Accordingly, the communication path 704 may be formed from any medium that is capable of transmitting a signal such as, for example, conductive wires, conductive traces, optical waveguides, or the like. In some embodiments, the communication path 704 may facilitate the transmission of wireless signals, such as WiFi, Bluetooth®, Near Field Communication (NFC) and the like. Moreover, the communication path 704 may be formed from a combination of mediums capable of transmitting signals. In one embodiment, the communication path 704 comprises a combination of conductive traces, conductive wires, connectors, and buses that cooperate to permit the transmission of electrical data signals to components such as processors, memories, sensors, input devices, output devices, and communication devices. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium.

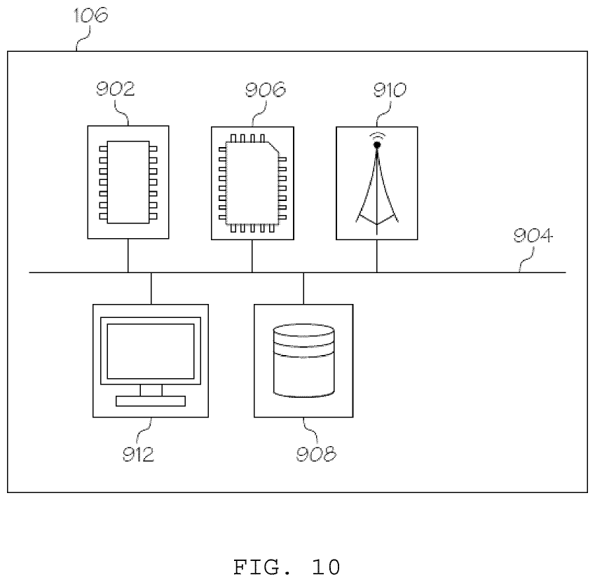

The ECU 626 includes one or more memory modules 706 coupled to the communication path 704. The one or more memory modules 706 may comprise RAM, ROM, flash memories, hard drives, or any device capable of storing machine readable and executable instructions such that the machine readable and executable instructions can be accessed by the one or more processors 702. The machine readable and executable instructions may comprise logic or algorithm(s) written in any programming language of any generation (e.g., 1GL, 2GL, 3GL, 4GL, or 5GL) such as, for example, machine language that may be directly executed by the processor, or assembly language, object-oriented programming (OOP), scripting languages, microcode, etc., that may be compiled or assembled into machine readable and executable instructions and stored on the one or more memory modules 706. Alternatively, the machine readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components. The memory modules 706 are discussed in more detail below in connection with FIG. 9.

The example ECU 626 includes the data storage component 708. The data storage component 708 may store data captured by the morphology analyzer 104 and/or received from the hematology analyzer 102, as disclosed in further detail below. The data storage component 708 may also store other data used by the various components of the ECU 626.

The ECU 626 comprises network interface hardware 710 for communicatively coupling the morphology analyzer 104 to the hematology analyzer 102 and/or the user interface device 106. This may allow data to be shared between the devices to improve the data collected by the devices, as disclosed herein.

In some embodiments, the ECU 626 comprises an output device 712. The output device 712 can include a graphical user interface (GUI), a screen, one or more devices in communication with the one or more processors 702 (such as smartphones, tables, and the like), and/or any other device or interface suitable for displaying data. In some examples, the output device 712, or another device, may be configured to as an input device to receive user input. The output device 712 may display images captured by the morphology analyzer 104 and/or data generated by the ECU 626.

Referring to FIGS. 8 and 9, the one or more memory modules 706 include a hematology data reception module 800, a confidence metric reception module 802, an image data reception module 804, and a data transmission module 806. Each of the hematology data reception module 800, the confidence metric reception module 802, the image data reception module 804, and the data transmission module 806 may be a program module in the form of operating systems, application program modules, and other program modules. Such a program module may include, but is not limited to, routines, subroutines, programs, objects, components, data structures and the like for performing specific tasks or executing specific data types as will be described below.

In some embodiments, the hematology data reception module 800 may receive data from the hematology analyzer 102 (FIG. 2). In particular, the hematology data reception module 800 may receive cell parameter data determined by the cell parameter determination module 404 (FIG. 4) and/or cell complexity data determined by the cell complexity determination module 408 (FIG. 4). The morphology analyzer 104 may utilize the data received from the hematology analyzer 102 to perform data analysis, as disclosed herein.

The confidence metric reception module 802 may receive confidence metrics from the hematology analyzer 102. In particular, the confidence metric reception module 802 may receive confidence level data determined by the confidence level determination module 406 (FIG. 4). The morphology analyzer 104 may utilize the confidence level data received by the confidence metric reception module 802 in conjunction with the hematology data received by the hematology data reception module 800 to perform data analysis, as disclosed herein.

The image data reception module 804 may receive images of a blood sample captured by the morphology analyzer 104. In particular, the image data reception module 804 may receive images captured by the imaging sensor 624 when a blood sample is illuminated by the fluorescent blue energy source 600, the fluorescent ultraviolet energy source 602, or the brightfield source.

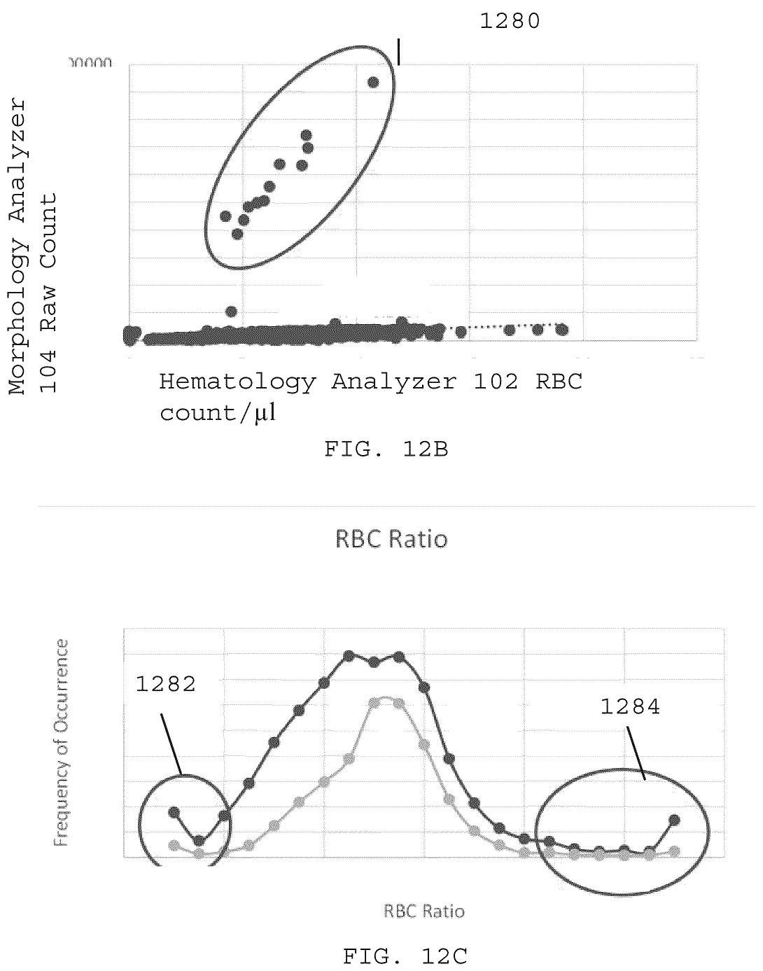

For example, as detailed below, FIG. 12A shows an example image 1100 of a blood sample that may be received by the image data reception module 804. The image 1100 in the example of FIG. 12A shows red blood cells and white blood cells. This image may be utilized to determine cell morphology, as disclosed herein.

Referring back to FIG. 9, the data transmission module 806 may transmit data generated by and/or received by the morphology analyzer 104 to the user interface device 106 (FIG. 1), as disclosed herein. In particular, the data transmission module 806 may cause the network interface hardware 710 to transmit the hematology data received by the hematology data reception module 800, the confidence metrics received by the confidence metric reception module 802, and the image data received by the image data reception module 804 to the user interface device 106. The user interface device 106 may perform further analysis, as disclosed in further detail below. In some examples, the hematology analyzer may transmit the hematology data, confidence metrics, and image data directly to the user interface device 106. In these examples, the data transmission module 806 need not transmit this data to the user interface device 106.

FIG. 10 schematically depicts an example configuration of the user interface device 106 of FIG. 1. In the illustrated example, the user interface device 106 includes one or more processors 902, a communication path 904, one or more memory modules 906, a data storage component 908, network interface hardware 910, and an output device 912, the details of which will be set forth in the following paragraphs.

Each of the one or more processors 902 may be any device capable of executing machine readable and executable instructions. Accordingly, each of the one or more processors 902 may be a controller, an integrated circuit, a microchip, a computer, or any other physical or cloud-based computing device. The algorithms, including the trained models, signal preprocessing, and noise removal methods discussed below, may be executed by the one or more processors 902. The one or more processors 902 are coupled to a communication path 904 that provides signal interconnectivity between various modules of the user interface device 106. Accordingly, the communication path 904 may communicatively couple any number of processors 902 with one another, and allow the modules coupled to the communication path 904 to operate in a distributed computing environment. Specifically, each of the modules may operate as a node that may send and/or receive data.

Accordingly, the communication path 904 may be formed from any medium that is capable of transmitting a signal such as, for example, conductive wires, conductive traces, optical waveguides, or the like. In some embodiments, the communication path 904 may facilitate the transmission of wireless signals, such as WiFi, Bluetooth®, Near Field Communication (NFC) and the like. Moreover, the communication path 904 may be formed from a combination of mediums capable of transmitting signals. In one embodiment, the communication path 904 comprises a combination of conductive traces, conductive wires, connectors, and buses that cooperate to permit the transmission of electrical data signals to components such as processors, memories, sensors, input devices, output devices, and communication devices. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium.

The user interface device 106 includes one or more memory modules 906 coupled to the communication path 904. The one or more memory modules 906 may comprise RAM, ROM, flash memories, hard drives, or any device capable of storing machine readable and executable instructions such that the machine readable and executable instructions can be accessed by the one or more processors 902. The machine readable and executable instructions may comprise logic or algorithm(s) written in any programming language of any generation (e.g., 1GL, 2GL, 3GL, 4GL, or 5GL) such as, for example, machine language that may be directly executed by the processor, or assembly language, object-oriented programming (OOP), scripting languages, microcode, etc., that may be compiled or assembled into machine readable and executable instructions and stored on the one or more memory modules 906. Alternatively, the machine readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components. The memory modules 906 are discussed in more detail below in connection with FIG. 10.

In embodiments, the user interface device 106 includes the data storage component 908. The data storage component 908 may store data received from the hematology analyzer 102 (FIG. 1) and/or the morphology analyzer 104 (FIG. 1), as disclosed in further detail below. The data storage component 908 may also store other data used by the various components of the user interface device 106.

In embodiments, the user interface device 106 comprises network interface hardware 910 for communicatively coupling the user interface device 106 to the hematology analyzer 102 (FIG. 1) and/or the morphology analyzer 104 (FIG. 1).

In some embodiments, the user interface device 106 comprises an output device 912. The output device 912 can include a graphical user interface (GUI), a screen, one or more devices in communication with the one or more processors 902 (such as smartphones, tables, and the like), and/or any other device or interface suitable for displaying data. In some examples, the output device 912, or another device, may be configured to as an input device to receive user input. The output device 912 may display images received from the morphology analyzer 104 and/or data generated by the user interface device 106.

Referring now to FIGS. 1, 10, and 11, the one or more memory modules 906 include a data reception module 1000, a cell classification module 1002, a cell parameter correction module 1004, and a data output module 1006. Each of the data reception module 1000, the cell classification module 1002, the cell parameter correction module 1004, and the data output module 1006 may be a program module in the form of operating systems, application program modules, and other program modules. Such a program module may include, but is not limited to, routines, subroutines, programs, objects, components, data structures and the like for performing specific tasks or executing specific data types as will be described below.

The data reception module 1000 may receive data from the hematology analyzer 102 and/or the morphology analyzer 104, as disclosed herein. In particular, the data reception module 1000 may receive cell parameter data determined by the cell parameter determination module 404 of the hematology analyzer 102, confidence metrics determined by the confidence level determination module 406 of the hematology analyzer 102, cell complexity data determined by the cell complexity determination module 408 of the hematology analyzer 102, and image data captured by the morphology analyzer 104. The data received by the data reception module 1000 may be used to perform data analysis, as disclosed herein.

The cell classification module 1002 may identify and classify the cells in the image of the blood sample captured by the morphology analyzer 104. These images may be received by the image data reception module 804 of the morphology analyzer 104 and transferred to the user interface device 106. In particular, the cell classification module 1002 may identify and classify the cells based on images of the blood sample. The cell classification module 1002 may classify the cells by type (e.g., red blood cells, white blood cells, and platelets) and morphology (e.g., disease state). In embodiments, machine learning techniques may be used to train the cell classification module 1002 to identify and classify the cells of the blood sample into cell type and morphology based on images of the blood sample. While described and depicted as residing within the memory modules 906 of the user interface device 106, it should be understood that the cell classification module 1002 may reside within the one or more memory modules 706 of the morphology analyzer 104, or any other suitable computing device such as a cloud computing device.

A system for image recognition of cell morphology may include one or more of the following processing steps: obtaining the image data of samples to be analyzed by capturing images, for example, using microscopy techniques; cleaning and preprocessing the acquired images to enhance the quality and remove any noise or artifacts (for example, using resizing, cropping, denoising, and normalization); segmenting the structures of interest from the background or other structures using various segmentation techniques such as thresholding, edge detection, region growing, or machine learning-based methods; extracting relevant features, such as shape, texture, intensity, or spatial properties, from the segmented structures using morphological operations, statistical analysis, and image texture analysis; and inputting the extracted features into a machine learning-based POC system to obtain, as output from the POC system, classification data.

The specific classification performed by the machine learning-based for morphology analysis depends on the diagnostic objective and the types of structures being analyzed. Some examples of common classifications in medical diagnostics include:
Normal vs. abnormal cells: The model distinguishes between normal cells and cells exhibiting abnormal morphology, such as cancerous or diseased cells. This classification helps identify potential pathological conditions.
Morphologic cell assessment:
   Detection of overall presentation of cell; and
   Detection of cell objects and features, such as nuclear features, vacuoles, organisms, inclusions, and the like.
Cell type identification: The model classifies cells into different cell types based on their morphology and/or size. For example, in blood cell analysis, the model can classify red blood cells, white blood cells, and platelets.
Subcellular component identification: The model identifies and classifies specific subcellular components or organelles within cells. This can include classifying nucleus, cytoplasm, mitochondria, or other cellular structures.
Differentiation of cell stages: The model determines the stage or maturation level of cells.
Parasite detection: The model detects eggs, ova, parasites, bacteria, or the like in a sample, such as an ear swab sample or fecal sample.
Disease-specific classification: The model classifies cells based on specific diseases or conditions. For example, in histopathology, the model can classify cells based on different types of cancer.

The algorithms residing in the cell classification module 1002 may utilize pre-trained machine-learning models to analyze the data and provide diagnostic information. Generally, these machine-learning models are developed (trained and tested) at centralized locations that collect large amounts of patient data from multiple sources (including, for example, multiple point of care systems) and have extensive computing resources to perform the necessary training and testing to generate the machine-learning models

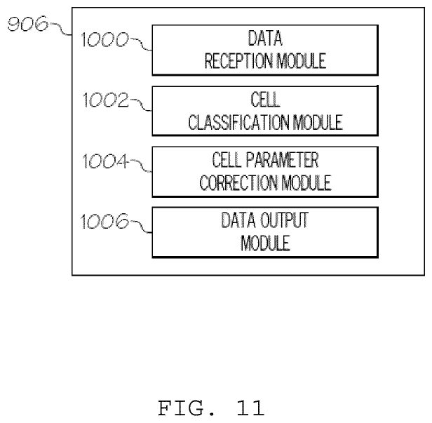

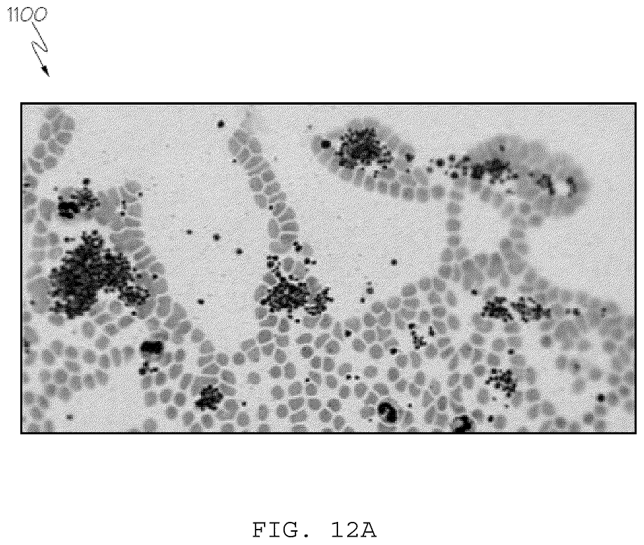

Referring still to FIG. 11, the cell parameter correction module 1004 may correct the cell parameters determined by the cell parameter determination module 404 of the morphology analyzer 104, based on the data received by data reception module 1000, as disclosed herein. While described and depicted as residing within the memory modules 906 of the user interface device 106, it should be understood that the cell parameter correction module 1004 may reside within the one or more memory modules 706 of the morphology analyzer 104, or any other suitable computing device such as a cloud computing device.

As described above, when morphologic abnormalities are present in a blood sample, the hematology analyzer 102 may have difficulty differentiating cell types. In some instances, the hematology analyzer 102 may flag or qualify certain parameters, indicating that the confidence level of the flagged parameter values is less than a threshold amount, and additional analysis should be performed to confirm or correct the values of those parameters. In embodiments, the user interface device 106 may confirm or correct the flagged parameter values, as disclosed herein.

In embodiments, the cell parameter correction module 1004 may determine the nature of the qualification specified by the hematology analyzer 102 and to evaluate the parameters that were qualified by the hematology analyzer 102. In some examples, the cell parameter correction module 1004 may explicitly correct the parameter values determined by the cell parameter determination module 404 of the hematology analyzer 102. In other examples, the cell parameter correction module 1004 may not change the values determined by the cell parameter determination module 404 but may provide added insight to the reason or name of a morphologic abnormality associated with the blood sample being evaluated (e.g., bands in the white cells, or spherocytes in the red cells). Specific examples of corrections that may be performed by the cell parameter correction module 1004 are described below. In embodiments, the cell parameter correction module 1004 may determine corrected parameter values using machine-learning logic applied to the image received by the image data reception module 804 and the cells identified and classified by the cell classification module 1002. In some examples, the cell parameter correction module 1004 may transmit the determined corrected parameter values to the hematology analyzer 102, as discussed above.

The data output module 1006 may output the image received by the image data reception module 804 and/or corrected values determined by the cell parameter correction module 1004. In some examples, the data output module 1006 may output a report showing corrected parameter values and an image captured by the morphology analyzer 104. FIGS. 12B, 13B, 14B and 15B show example reports that may be output by the data output module 1006, which are discussed in further detail below.

However, in some instances, the hematology analyzer 102 may fail to flag or qualify inaccurate parameters. For example and as noted above, left shift and SP-RBC is difficult to identify with a hematology analyzer 102 alone, and they lack clear thresholds. Likewise, hematology analyzers 102 may fail to identify clumped platelets, inaccurately identifying the clumped platelets as white cells. Similarly, in instances in which un-lysed RBC appear on the white cell dot plot (e.g., FIG. 6B) and cannot be distinguished from lymphocytes, the hematology analyzer 102 may fail to identify the lysing error. In these examples, the hematology analyzer 102 may fail to flag or qualify parameters. Nonetheless, results from the morphology analyzer 104 may be used to correct the inaccurate parameters.

As noted above, one morphologic abnormality that may occur is when platelets clump together. This condition can make it difficult to determine the true number of platelets in a blood sample if the number of PLT in each clump cannot be determined. In particular, it can be difficult for the hematology analyzer 102 to distinguish between large PLT and PLT clumps and between PLT clumps and white cells. As such, when PLT are clumped, the number of PLT determined by the hematology analyzer 102 is generally an underrepresentation of the true number of PLT in the sample.

This can be a concern if a patient is being prepared for surgery and a hematology analysis is performed to confirm that there are adequate PLT for clotting after surgery. There is a minimum number of PLT, typically 100-150 K/μl, that a veterinarian expects to perform surgery, where a normal PLT count could be between 300-500 K/μl for dogs and cats. If PLT are clumped and the PLT value reported by the hematology analyzer 102 is artificially below this threshold, it may result in surgery not being performed even though there are actually enough PLT present for the surgery to go forward.

The hematology analyzer 102 can use techniques, such as flow cytometry technology, to identify if clumped PLT are likely present in a blood sample. As such, in the example of FIG. 5, the PLT value of 90 K/μl is flagged with a “*” and a notation is made indicating that a likely clumped PLT has been identified. Furthermore, the values of MPV, PCT, and PDW associated with platelets are also flagged with “*” Without being bound by theory, in some circumstances hematology analyzers using flow cytometry, impedance, fluorescence, and the like are able to identify the likely presence of clumped PLT, but are not able to definitely identify or correct for this condition.

The user interface device 106 may be used to accurately count the PLT using the morphology analyzer 104. In particular, the cell parameter correction module 1004 may count the number of PLT in the image received by the image data reception module 804 and either confirm or correct the PLT count received by the hematology data reception module 800 from the hematology analyzer 102.

The cell parameter correction module 1004 may also use the data received by the data reception module 1000 for calibration, as disclosed herein. Without the data from the hematology analyzer 102, the morphology analyzer 104 may only be able to determine PLT per field of view, which is not in itself indicative of PLT per μl of fluid. However, the hematology data reception module 800 and/or the data reception module 1000 may receive a red blood cell count per μl from the hematology analyzer 102. The cell parameter correction module 1004 may then count the number of red blood cells in the image received by the image data reception module 804. The cell parameter correction module 1004 may then determine a calibration value comprising a ratio between the red blood cell count per μl and the red blood cell count per field of view. The cell parameter correction module 1004 may use the calibration value to convert the PLT per field of view to PLT per μl. In this way, utilizing a known count of RBC per μl of fluid from the hematology analyzer 102, a known count of RBC per field of view from the morphology analyzer 104, and a known count of PLT per field of view from the morphology analyzer 104, an estimate of the PLT per μl of fluid can be estimated. The cell parameter correction module 1004 may similarly use the RBC-derived calibration value to correct values associated with white blood cells.

In embodiments and referring to FIG. 12B, counts of RBC per μl of fluid are depicted on the x-axis, and RBC counts calculated by the morphology analyzer 104 are shown on the y-axis. The example data depicted in FIG. 12B shows the results of numerous runs performed on the hematology analyzer 102 and the morphology analyzer 104. As shown, in most circumstances, the calculated RBC count from the morphology analyzer has a generally linear relationship with the counts of RBC per μl of fluid. Outliers, e.g., circled results 1280 show significantly higher RBC counts on the morphology analyzer 104 than expected, which can result from a number of conditions, such as and without limitation, improper preparation and dilution of the sample and the like. In embodiments, upon receiving a calculated RBC count from the morphology analyzer 104 that exceeds a configurable threshold (e.g., outside of the linear relationship depicted), the hematology system 100 provides an alarm indicating that another sample should be prepared and run on the morphology analyzer 104 and/or the hematology analyzer 102.

Similarly and referring to FIG. 12C, RBC ratios provided the hematology analyzer 102 and morphology analyzer 104 are shown. The RBC ratio depicted indicates the RBC value provided by the hematology analyzer 102 divided by the RBC value calculated by the morphology analyzer 104. As shown across multiple samples, the RBC ratios generally follow a bell-curve distribution. However, outliers 1282 and 1284 are anomalies that depart from the bell-curve distribution. In embodiments, upon calculating an RBC ratio that exceeds a configurable threshold (e.g., outside of bell-shaped curve as depicted), the hematology system 100 provides an alarm indicating that another sample should be prepared and run on the morphology analyzer 104 and/or the hematology analyzer 102. In this way, incorrect RBC values from the morphology analyzer 104 can be detected.

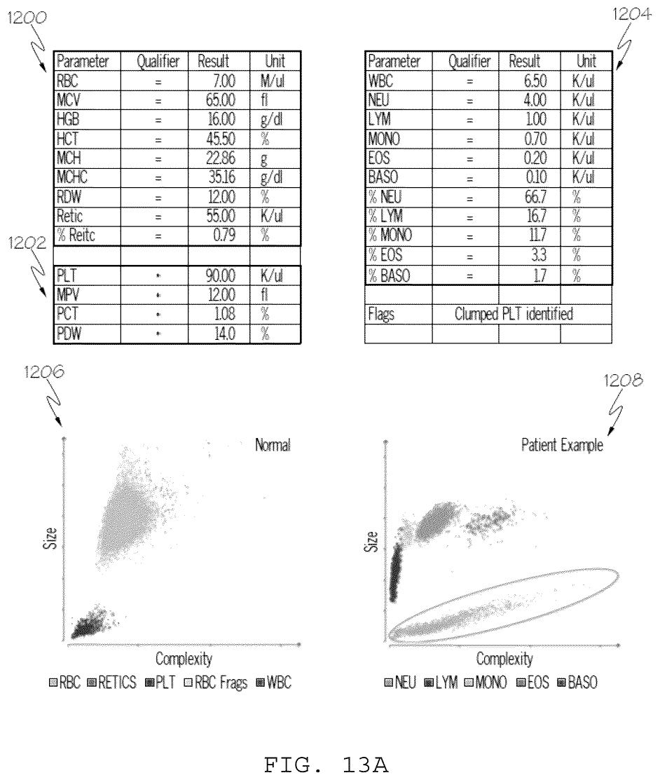

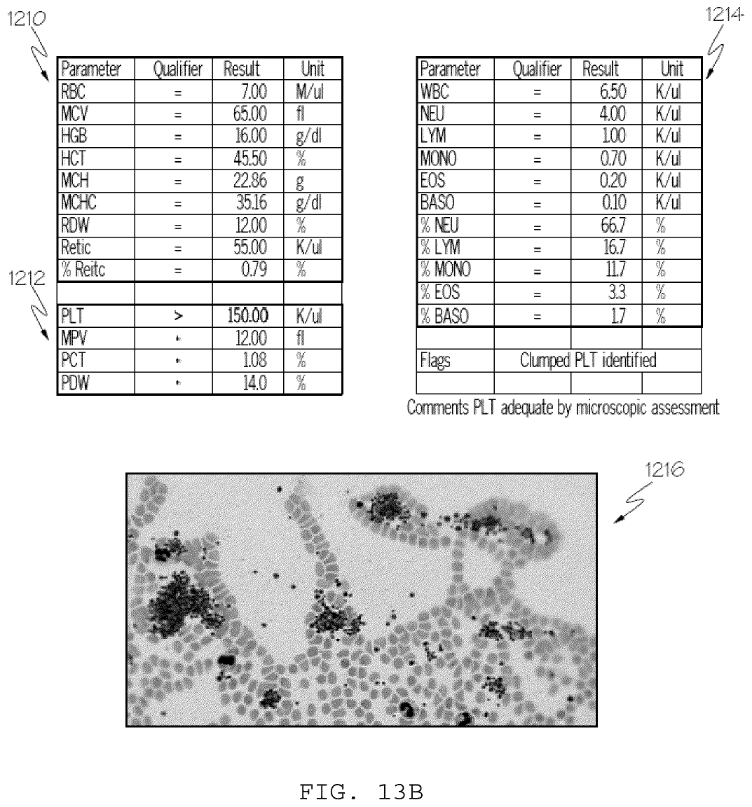

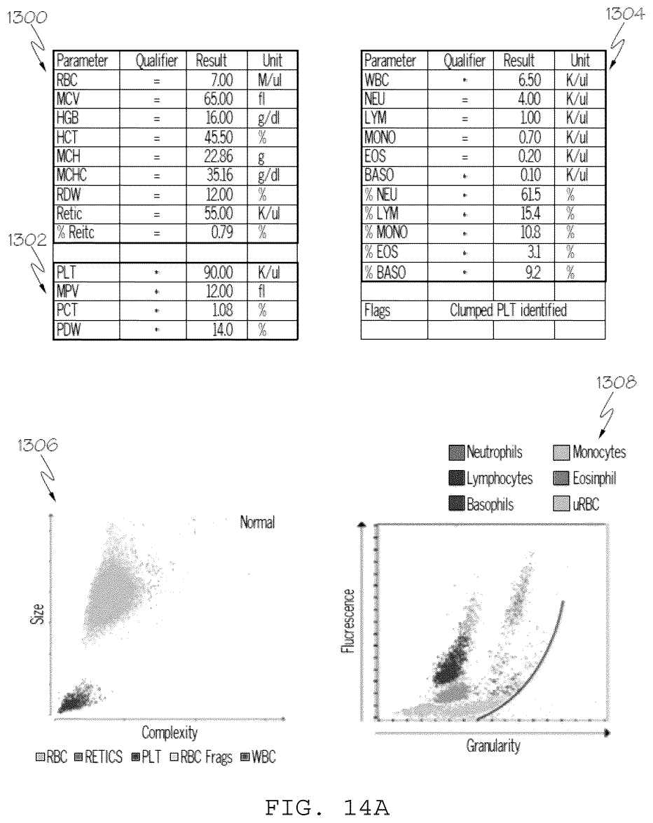

After the cell parameter correction module 1004 determines a corrected PLT count, the data output module 1006 may output a report showing a corrected PLT value. In some examples, rather than outputting a corrected value, the data output module 1006 may output a report showing that the PLT value is greater than a predetermined threshold. For example, in some instances, such as pre-surgery screening, an estimated PLT value is unnecessary, and it is sufficient to determine that the PLT value is above the predetermined threshold. FIG. 13A shows an example report that may be generated by the user interface device 106, and FIG. 13B shows a report with corrected data that may be output by the data output module 1006.

In the example of FIG. 13A, a first section 1200 shows information about red blood cells, a second section 1202 shows information about platelets, and a third section 1204 shows information about white blood cells. FIG. 13A also includes dot plots 1206 and 1208, which show information about cell complexity. In the example of FIG. 13A, the PLT value shown is 90 K/μl, which is less than the threshold of 150 K/μl required for surgery, as discussed above. However, in the example of FIG. 13A, the PLT value is qualified with “*” indicating that the confidence level for this value is below a predetermined threshold value. While qualified in FIG. 13A, it should be understood that in some circumstances, the hematology analyzer 102 may report an inaccurate PLT count without qualification.

FIG. 13B shows a report that may be output by the data output module 1006 after the cell parameter correction module 1004 corrects the PLT value. In the example of FIG. 13B, a first section 1210 shows information about red blood cells, a second section 1212 shows information about platelets, and a third section 1214 shows information about white blood cells. FIG. 13B also includes an image that may be captured by the morphology analyzer 104. In the example of FIG. 13B, the PLT is indicated as being greater than 150 K/μl, which is greater than the threshold required for performing surgery. As such, a physician or veterinarian viewing the example report of FIG. 13B would be able to perform surgery on the patient associated with the tested blood sample.

In the example of FIG. 13A, only the parameters associated with PLT were inaccurate. As such, the values associated with white blood cells and red blood cells determined by the hematology analyzer 102 are simply re-reported by the data output module 1006, as shown in FIG. 13B. Furthermore, FIG. 13B shows an indication that PLT is adequate by microscopic assessment (i.e., PLT>150 K/μl). In embodiments, PLT are reported in a semi-quantitative manner if the platelets are adequate (within reference interval, i.e., PLT>150 K/μl), mildly reduced, moderately reduced, or markedly reduced. These four groupings provide actionable regions for the customer to consider the patients ability to clot and to develop an appropriate clinical plan.

However, in some examples, clumped PLT impacts not only the ability of the hematology analyzer 102 to accurately count PLT, but also the ability of the hematology analyzer 102 to accurately count white cells, since the PLT clumps may have features that present in a manner similar to white cells. When this occurs, the user interface device 106 may be used to correct parameters associated with white cells. In particular, the proportion of each of the 5-types of white cells (NEU, LYM MONO, EOS, BASO) may be used to correct the differential on the report output by the data output module 1006. Based on an evaluation of the dot plot generated by the cell complexity determination module 408, WBC may be impacted if clumped PLT are affecting white cells and could be counted as white cells by the hematology analyzer 102. In some examples, a corrected white count may not be available, but a semi-quantitative assessment of WBC may be determined by the user interface device 106 and provided on the report output by the data output module 1006. If part of the differential is confirmed to be corrected on the dot plot, then the relative proportion of cells from the morphology % Diff calculation can be used to back- calculate the estimated true WBC value.

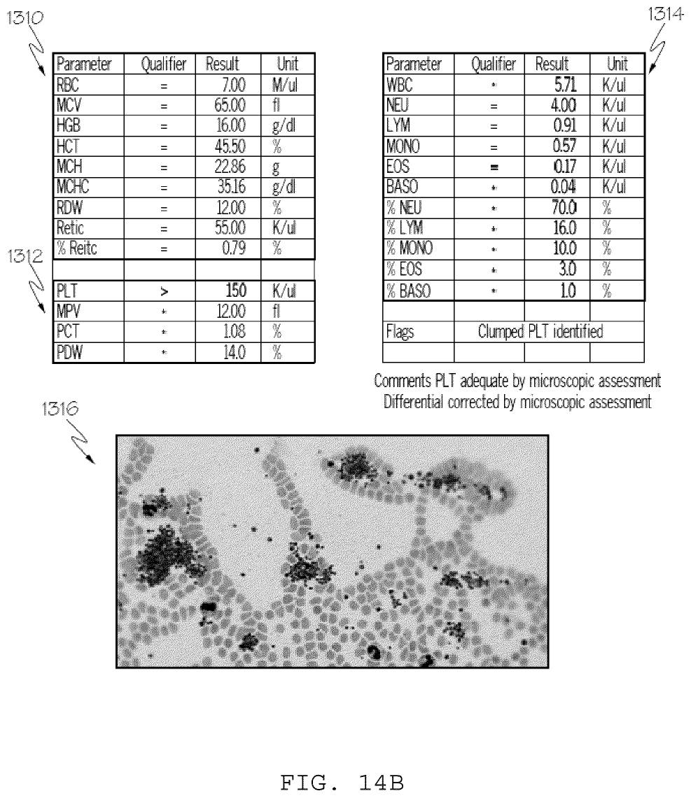

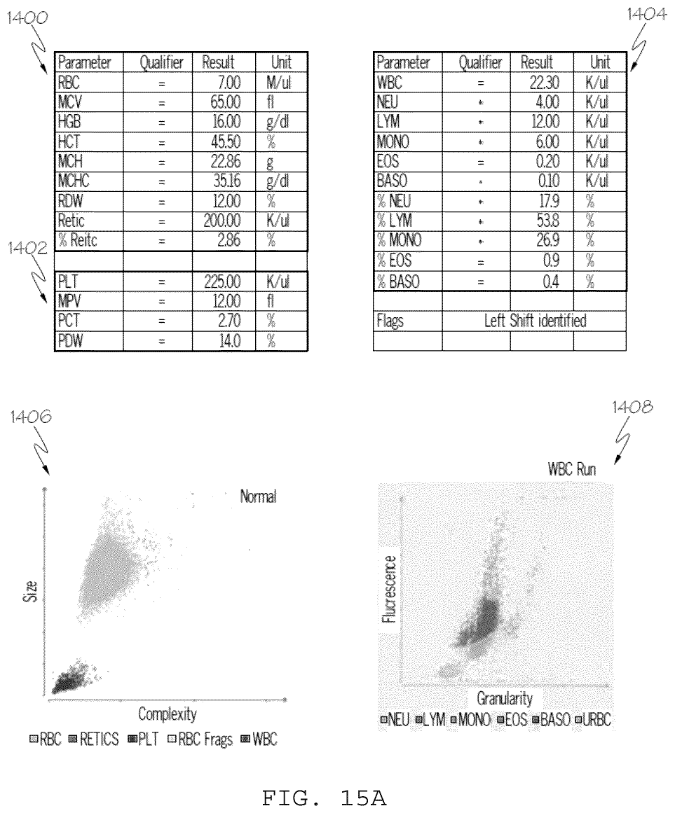

FIG. 14A shows an example report that may be generated by the hematology analyzer 102. In the example of FIG. 14A, a first section 1300 shows information about red blood cells, a second section 1302 shows information about platelets, and a third section 1304 shows information about white blood cells. FIG. 14A also includes dot plots 1306 and 1308, which show information about cell complexity. In the example of FIG. 14A, the PLT and the WBC values are qualified with “*,” indicating that their confidence level is below a predetermined threshold. While qualified in FIG. 14A, it should be understood that in some circumstances, the hematology analyzer 102 may report an inaccurate PLT and WBC without qualification.

FIG. 14B shows a report that may be output by the data output module 1006 with corrected values, as determined by the user interface device 106. In the example of FIG. 14B, a first section 1310 shows information about red blood cells, a second section 1312 shows information about platelets, and a third section 1314 shows information about white blood cells. FIG. 14B also includes an image 1316 that may be captured by the morphology analyzer 104. In the example of FIG. 14B, the corrected PLT is indicated as being greater than 150 K/μl and the parameter values associated with white blood cells are also corrected.

Another morphologic abnormality that may be present in a blood sample is Left Shift. Left Shift is a condition where an inflammatory condition presents with an elevated number of immature NEU (bands, etc.) and/or toxic NEU. These cell types can move the cluster population of NEU and interfere with the ability to separate from MONO and LYM. When this happens, NEU, LYM, and MONO can all be qualified, and a Left Shift flag is identified.

The morphology analyzer 104 may then use microscopy techniques to evaluate the differential similarity to the clumped PLT with differential assessment, as discussed above, but can also include bands and toxic NEU in the assessment. The morphology analyzer 104 may update these values, while other parameters would remain the same. The specific outcome is a potential adjustment to the differential as well as a morphologic identification of the type and relative concentration of bands and/or toxic NEU. WBC is generally not impacted by Left Shift, and the only difficulty is separating the different white cell types. As such, there is no correction to WBC and the absolute and percentage differentials can be updated.

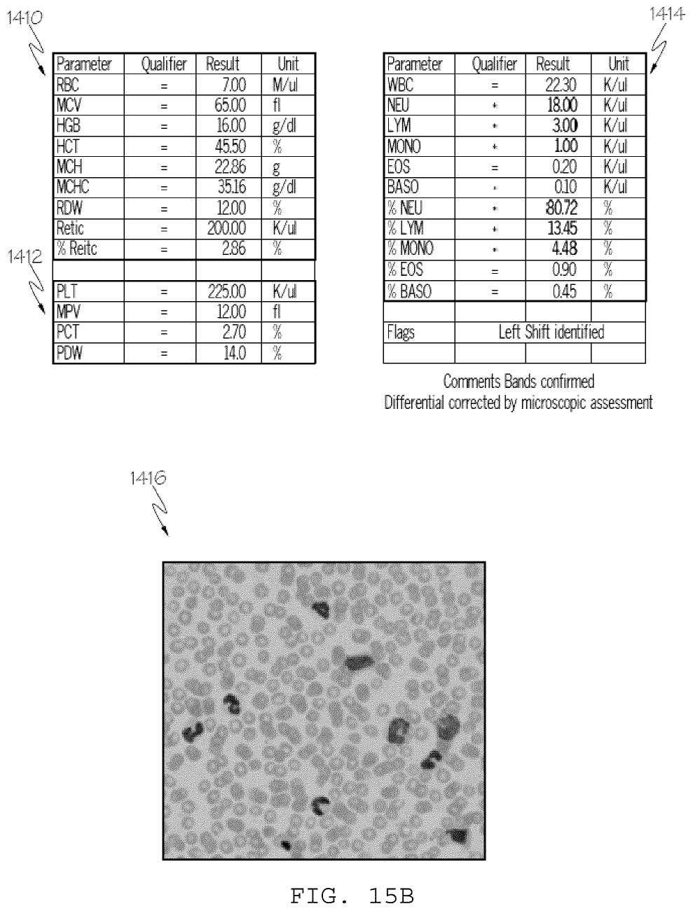

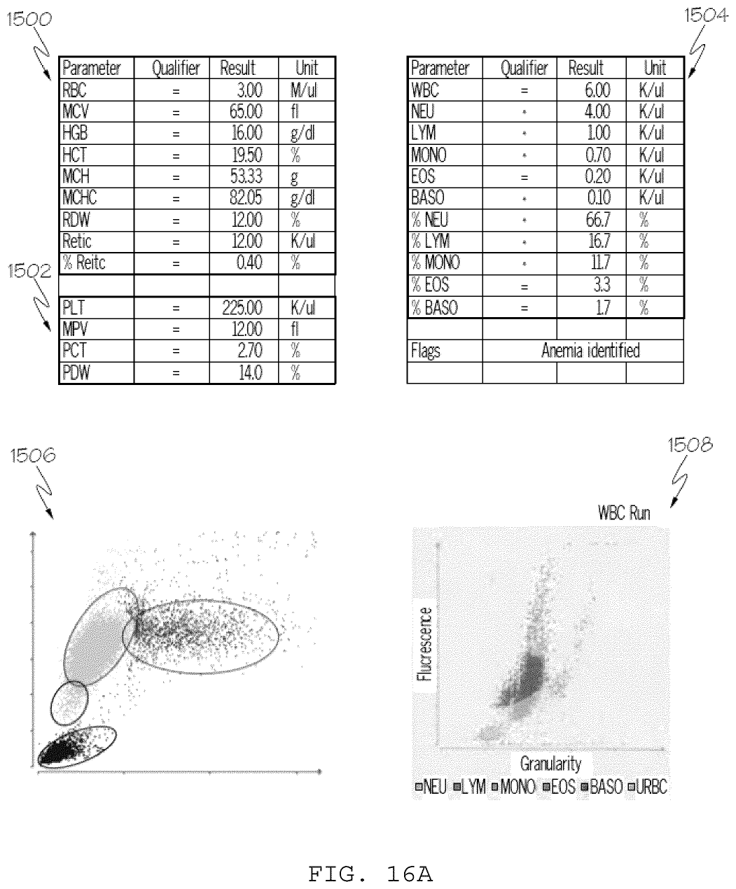

FIGS. 15A and 15B show a report that may be generated by the hematology analyzer 102 and a report that may be generated by the user interface device 106, respectively, when Left Shift is present. In the example of FIG. 15A, a first section 1400 shows information about red blood cells, a second section 1402 shows information about platelets, and a third section 1404 shows information about white blood cells. FIG. 15A also includes dot plots 1406 and 1408, which show information about cell complexity. In the example of FIG. 15B, a first section 1410 shows information about red blood cells, a second section 1412 shows information about platelets, and a third section 1414 shows revised information about white blood cells. Similar to the example shown above in FIGS. 14A and 14B, the revised information about the white blood cells can be determined by the morphology analyzer 104, and the revised information displayed via the user interface device 106. FIG. 15B also includes an image 1416 that may be captured by the morphology analyzer 104. While in FIG. 15A, information about the white cells in section 1404 show qualifications, it should be understood that this is an example, and in some instances, the hematology analyzer 102 may report inaccurate information without qualification.

Another morphologic abnormality that may be present in a blood sample is SP-RBC. The hematology analyzer 102 can identify pathologic red blood cells as part of the red blood cell analysis. The outcome of this assessment is not a concern around the count of cells in the sample, but with the type of RBC morphology present. There may be many potential reasons for small pathologic red blood cells and determining the type of abnormality in morphology can lead to different diagnostic paths and treatments. In embodiments, when small pathologic red blood cells are present, the data output module 1006 may output the parameter values determined by the hematology analyzer 102 along with comments indicating the type of RBC morphology identified. However, as noted above, in some circumstances, the data output module 1006 may not correctly identify SP-RBC, and comments may not be provided by the hematology analyzer 102.

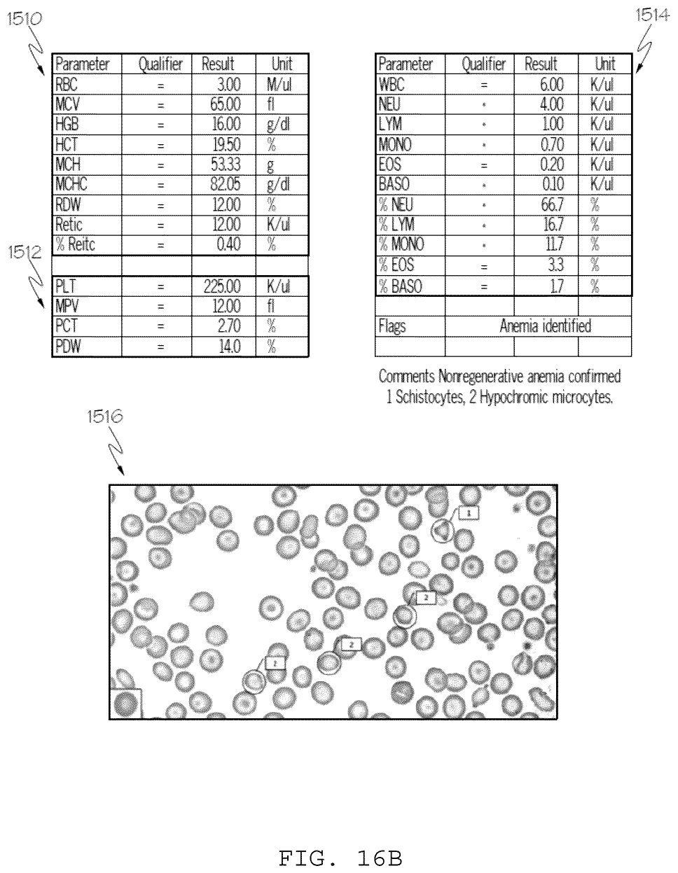

FIG. 16A shows an example report that may be generated by the hematology analyzer 102 when anemia is identified, and FIG. 16B shows an example report that may be output by the data output module 1006 indicating identified RBC morphology. In the example of FIG. 16A, a first section 1500 shows information about red blood cells, a second section 1502 shows information about platelets, and a third section 1504 shows information about white blood cells. FIG. 16A also includes dot plots 1506 and 1508, which show information about cell complexity. In the example of FIG. 16B, a first section 1510 shows information about red blood cells, a second section 1512 shows information about platelets, and a third section 1514 shows information about white blood cells. FIG. 16B also includes an image 1516 that may be captured by the morphology analyzer 104.

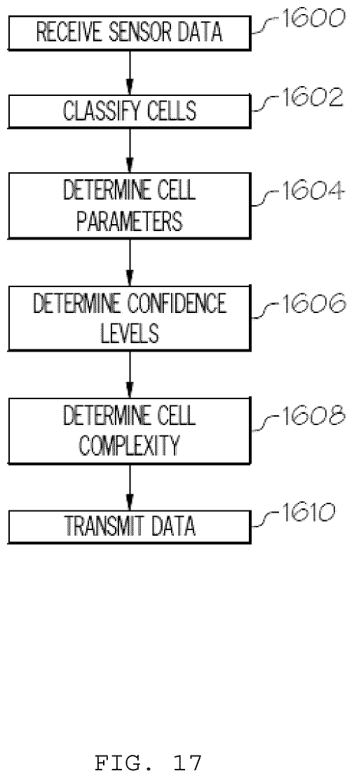

FIG. 17 depicts a flowchart of an example method for operating the hematology analyzer 102, according to the embodiments disclosed herein.

At step 1600, the energy source 210 illuminates a blood sample and the sensor data reception module 400 receives energy signal data from the sensors 225, 230, 232, and 235 based on the illumination of the cells of the blood sample by the energy source 210. At step 1602, the cell classification module 402 classifies the cells of the blood sample based on the data received by the sensor data reception module 400. At step 1604, the cell parameter determination module 404 determines parameters associated with the cells of the blood sample based on the data received by the sensor data reception module 400. At step 1606, the confidence level determination module 406 determines confidence levels associated with the parameters determined by the cell parameter determination module 404. At step 1608, the cell complexity determination module determines complexity of the cells of the blood sample based on the data received by the sensor data reception module 400. At step 1610, the hematology analyzer 102 transmits the determined cell classifications, cell parameter values, confidence levels, and cell complexity to the morphology analyzer 104.

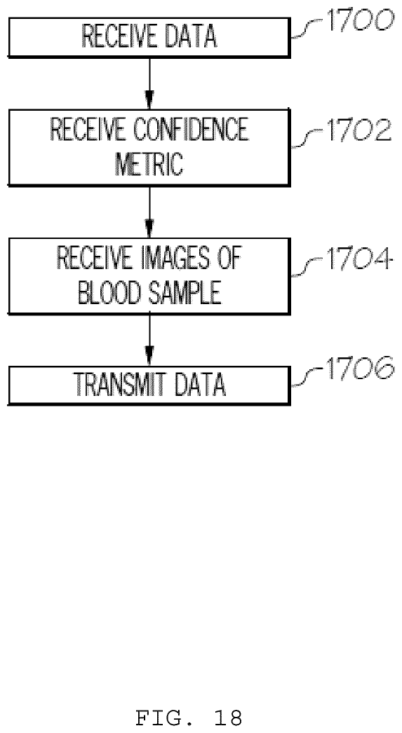

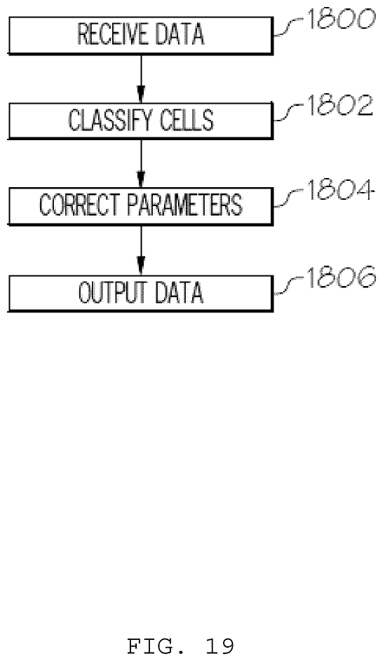

FIG. 18 depicts a flowchart of an example method for operating the morphology analyzer 104, according to the embodiments disclosed herein. At step 1700, the hematology data reception module 800 receives data from the hematology analyzer 102. The received data may include cell classifications, cell parameter values, and cell complexity, as determined by the cell classification module 402 and/or the cell parameter determination module 404. At step 1702, the confidence metric reception module 802 receives confidence levels associated with the received cell parameter values, as determined by the confidence level determination module 406.

At step 1704, the image data reception module 804 receives one or more images of a blood sample captured by the imaging sensor 624. At step 1706, the data transmission module 806 transmits data generated by and/or received by the morphology analyzer 104 to the user interface device 106. In particular, the data transmission module 806 may cause the network interface hardware 710 to transmit the hematology data received by the hematology data reception module 800, the confidence metrics received by the confidence metric reception module 802, and the image data received by the image data reception module 804 to the user interface device 106.

FIG. 19 depicts a flowchart of an example method for operating the user interface device 106, according to the embodiments disclosed herein.

At step 1800, the data reception module 1000 receives data from the hematology analyzer 102 and/or the morphology analyzer 104. In particular, the data reception module 1000 may receive cell parameter data determined by the cell parameter determination module 404 of the hematology analyzer 102, confidence metrics determined by the confidence level determination module 406 of the hematology analyzer 102 and/or cell complexity data determined by the cell complexity determination module 408 of the hematology analyzer 102. The data reception module 1000 may also receive image data captured by the morphology analyzer 104.

At step 1802, the cell classification module 1002 identifies and classifies the cells of the blood sample by cell type and morphology based on the received images. At step 1804, the cell parameter correction module 1004 corrects one or more of the received parameter values based on the one or more images received by the image data reception module 804. In particular, in some instances parameter values having a confidence level below a predetermined threshold are corrected by the cell parameter correction module 1004. In instances in which the confidence level is above the predetermined threshold, the parameter values may nonetheless be corrected by the cell parameter correction module 1004 based on the parameter values from the image data reception module 804.

In some examples, the cell parameter correction module 1004 may also determine one or more morphologies associated with the blood samples based on the image received by the image data reception module 804.

At step 1806, the data output module 1006 outputs the corrected parameter values determined by the cell parameter correction module 1004. The data output module 1006 may also output parameter values received by the data reception module 1000 for parameters that were not corrected.

In the example of clumped platelets, as noted above, the data output module 1006 can output corrected parameter values regardless of whether the hematology analyzer 102 had identified clumped platelets. However, even in circumstances when the hematology analyzer 102 can identify the presence of clumped platelets, hematology analyzers using flow cytometry, impedance, florescence and the like, it is difficult count the number of platelets in a clump. By contrast, the morphology analyzer 104 counts the number of platelets in the platelet clumps to provide the corrected parameter values. The corrected parameter values, in embodiments, are reported in a semi-quantitative manner if the platelets are adequate (within reference interval, i.e., PLT>150 K/μl), mildly reduced, moderately reduced, or markedly reduced. These four groupings provide actionable regions for the customer to consider the patients ability to clot and to develop an appropriate clinical plan. Moreover, in embodiments, the semi-quantitative PLT values are provided to the algorithm update module 412 (FIG. 4) of the hematology analyzer 102 and the cell parameter determination module 404 (FIG. 4) is changed so the hematology analyzer 102 can better identify the presence of clumped platelets.

In the example of left shift, the data output module 1006 can output corrected parameter values regardless of whether the hematology analyzer 102 had identified left shift. As noted above, in hematology analyzers using flow cytometry, impedance, florescence and the like, it is difficult to appropriately identify left shift. By contrast, the morphology analyzer 104, by interrogating cell morphology can identify left shift as well as indications if the left shift is attributable to bands, toxic change, and/or other neutrophil precursors. provide the corrected parameter values. Moreover, in embodiments, the identification of and correction of parameters associated with left shift are provided to the algorithm update module 412 (FIG. 4) of the hematology analyzer 102 and the cell parameter determination module 404 (FIG. 4) is changed to so the hematology analyzer 102 can better identify left shift.

In the example of SP-RBC, the data output module 1006 can output corrected parameter values regardless of whether the hematology analyzer 102 had identified SP-RBC. As noted above, in hematology analyzers using flow cytometry, impedance, florescence and the like, it is difficult to appropriately identify SP-RBC. By contrast, the morphology analyzer 104, by interrogating cell morphology can identify SP-RBC as well as the morphologies as a percent of RBC in semi-quantitative buckets. In embodiments, the morphology analyzer 104 may provide correction of WBC and differential values. Moreover, in embodiments, the identification of and correction of parameters associated with SP-RBC are provided to the algorithm update module 412 (FIG. 4) of the hematology analyzer 102 and the cell parameter determination module 404 (FIG. 4) is changed to so the hematology analyzer 102 can better identify SP-RBC and the associated morphologies.

In the case of un-lysed RBC, as noted above, the un-lysed RBC The hematology analyzer 102 cannot distinguish the un-lysed RBC from lymphocytes and the hematology analyzer 102 may fail to identify the lysing error. By contrast, because the morphology analyzer 104 does not require the use of lysing agents, the sample interrogated by the morphology analyzer 104 will have significantly fewer lymphocytes than expected by the corresponding interrogation of the sample by the hematology analyzer. In these circumstances, the data output module 1006 may provide corrected parameters of lymphocytes from the morphology analyzer 104 and/or provide an alert that the analysis of the hematology analyzer 102 was insufficient and should be re-performed.

It should now be understood that embodiments disclosed herein are directed to a point-of-care hematology analyzer. By combining results form a hematology analyzer, such as a flow cytometer, and a morphology analyzer, such as a digital microscope, more accurate data can be presented to patients and clinicians than would be possible by using either device alone. As such, the embodiments disclosed herein allow for improved point-of-care analysis.

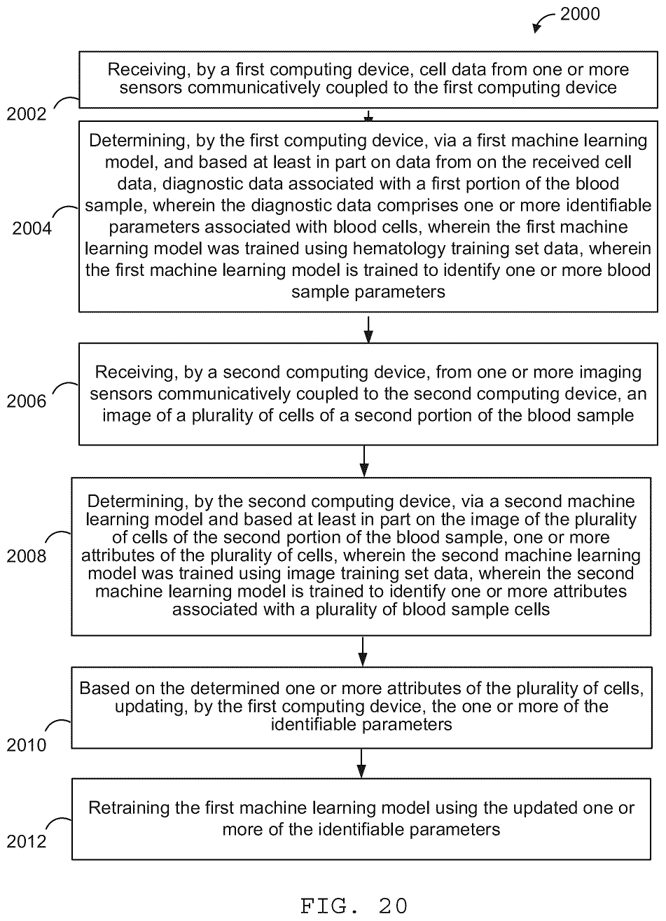

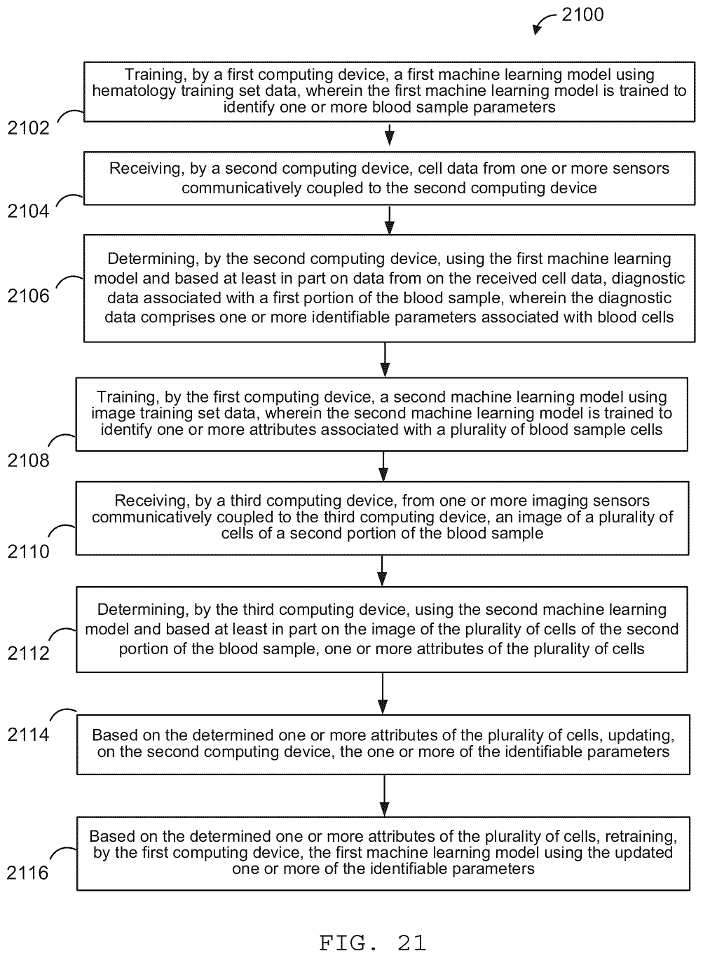

Now referring to FIG. 20, an example method is illustrated. Method 2000 shown in FIG. 20 presents an example of a method that could be used with the components shown in FIGS. 1-11, for example. Further, devices or systems may be used or configured to perform logical functions presented in FIG. 20. In other examples, components of the devices and/or systems may be arranged to be adapted to, capable of, or suited for performing the functions, such as when operated in a specific manner.

Method 2000 may include one or more operations, functions, or actions as illustrated by one or more of blocks 2002-2012. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 2002, method 2000 for receiving, by a first computing device, cell data from one or more sensors communicatively coupled to the first computing device.

In some examples, the one or more identifiable parameters associated with blood cells comprises one or more identifiable parameters associated with red blood cells. In some examples, the one or more identifiable parameters associated with red blood cells comprises one or more of the following: (i) total red blood count (RBC), (ii) mean corpuscular volume (MCV), (iii) hemoglobin (HGB), (iv) hematocrit (HCT), (v) mean corpuscular hemoglobin (MCH), (vi) mean corpuscular hemoglobin concentration (MCHC), (vii) red distribution width (RDW), (viii) reticulocyte count (Retic), (ix) percentage of reticulocyte (% Retic), (x) platelet count (PLT), (xi) mean platelet volume (MPV), (xii) plateletcrit (PCT), and (xiii) platelet distribution width (PDW).

In some examples, the one or more identifiable parameters associated with blood cells comprises one or more identifiable parameters associated with white blood cells. In some examples, the one or more identifiable parameters associated with white blood cells comprises one or more of the following: (i) white blood count (WBC), (ii) absolute neutrophil count (NEU), (iii) absolute lymphocyte count (LYM), (iv) absolute monocyte count (MONO), (v) absolute eosinophil count (EOS), (vi) absolute basophil count (BASO), (vii) percentage neutrophils (% NEU), (viii) percentage lymphocytes (% LYM), (ix) percentage monocytes (% MONO), (x) percentage absolute eosinophils (% EOS), and (xi) percentage basophils (% BASO).

In some examples, the first computing device comprises a hematology analyzer. In some examples, the first computing device further comprises a cloud-based modeling computing device. In some examples, the hematology analyzer comprises a flow cytometer.

At block 2004, method 2000 involves, determining, by the first computing device, via a first machine learning model, and based at least in part on data from on the received cell data, diagnostic data associated with a first portion of the blood sample, wherein the diagnostic data comprises one or more identifiable parameters associated with blood cells, wherein the first machine learning model was trained using hematology training set data, wherein the first machine learning model is trained to identify one or more blood sample parameters.

In examples, determining the diagnostic data comprises evaluating detected cell size and detected cell complexity. In some examples, determining the diagnostic data comprises evaluating detected cell size and detected fluorescence.

At block 2006, method 2000 involves, receiving, by a second computing device, from one or more imaging sensors communicatively coupled to the second computing device, an image of a plurality of cells of a second portion of the blood sample.

In some examples, the second computing device comprises a morphology analyzer. In some examples, the morphology analyzer comprises one or more morphology processors communicatively coupled to the one or more imaging sensors, one or more energy sources optically coupled to the one or more imaging sensors, and an objective lens optically coupled to the one or more imaging sensors. In some examples, if the stain intensity is relatively low, modifying the intensity of the light source includes decreasing the intensity of the light source.

At block 2008, method 2000 involves determining, by the second computing device, via a second machine learning model and based at least in part on the image of the plurality of cells of the second portion of the blood sample, one or more attributes of the plurality of cells, wherein the second machine learning model was trained using image training set data, wherein the second machine learning model is trained to identify one or more attributes associated with a plurality of blood sample cells.

In some examples, determining the one or more attributes of the plurality of cells comprises identifying a subset of the plurality of cells having a cell size or a cell morphology associated with left shift. In some examples, retraining the first machine learning model using the updated one or more of the identifiable parameters comprises retraining the first machine learning model in response to identifying the subset of the plurality of cells having the cell size of the cell morphology associated with left shift. In some examples, determining the one or more attributes of the plurality of cells comprises identifying a subset of the plurality of cells having a cell size or a cell morphology associated with small pathologic red blood cells. In some examples, retraining the first machine learning model using the updated one or more of the identifiable parameters comprises retraining the first machine learning model in response to identifying the subset of the plurality of cells having the cell size of the cell morphology associated with small pathologic red blood cells. In some examples, determining the one or more attributes of the plurality of cells comprises identifying individual platelet clumps, and in response to identifying the individual platelet clumps, counting a number of individual platelets in the individual platelet clumps. In some examples, retraining the first machine learning model using the updated one or more of the identifiable parameters comprises retraining the first machine learning model based at least in part on the counted number of individual platelets.

At block 2010, method 2000 involves, based on the determined one or more attributes of the plurality of cells, updating, by the first computing device, the one or more of the identifiable parameters.

At block 2012, method 2000 involves retraining the first machine learning model using the updated one or more of the identifiable parameters.

In some examples, method 2000 further includes, comprising emitting a beam of energy with an energy source to impinge cells of the first portion of the first portion of the blood sample within a cuvette.

In some examples, determining the one or more attributes of the plurality of cells comprises determining a number of lymphocytes in the second portion of the blood sample, and wherein the method 2000 further includes, wherein training the one or more machine learning models comprises, based on inputting the one or more training images into the machine learning model, (i) predicting, by the one or more machine learning models, an outcome of a determined condition of the one or more training images, (ii) comparing the at least one outcome to the characteristic of the one or more training images, and (iii) adjusting, based on the comparison, the machine learning model.

In some examples, method 2000 further includes transmitting, by the first computing device, a treatment plan based on the updated one or more of the identifiable parameters.

In one aspect, a non-transitory computer-readable medium, having stored thereon program instructions that, when executed by one or more processors, cause the one or more processors to perform a set of operations comprises (i) receiving, by a first computing device, cell data from one or more sensors communicatively coupled to the first computing device; (ii) determining, by the first computing device, via a first machine learning model, and based at least in part on data from on the received cell data, diagnostic data associated with a first portion of the blood sample, wherein the diagnostic data comprises one or more identifiable parameters associated with blood cells, wherein the first machine learning model was trained using hematology training set data, wherein the first machine learning model is trained to identify one or more blood sample parameters; (iii) receiving, by a second computing device, from one or more imaging sensors communicatively coupled to the second computing device, an image of a plurality of cells of a second portion of the blood sample; (iv) determining, by the second computing device, via a second machine learning model and based at least in part on the image of the plurality of cells of the second portion of the blood sample, one or more attributes of the plurality of cells, wherein the second machine learning model was trained using image training set data, wherein the second machine learning model is trained to identify one or more attributes associated with a plurality of blood sample cells; (v) based on the determined one or more attributes of the plurality of cells, updating, by the first computing device, the one or more of the identifiable parameters; and (vi) retraining the first machine learning model using the updated one or more of the identifiable parameters.

Now referring to FIG. 21, an example method is illustrated. Method 2100 shown in FIG. 21 presents an example of a method that could be used with the components shown in FIGS. 1-11, for example. Further, devices or systems may be used or configured to perform logical functions presented in FIG. 21. In other examples, components of the devices and/or systems may be arranged to be adapted to, capable of, or suited for performing the functions, such as when operated in a specific manner.

Method 2100 may include one or more operations, functions, or actions as illustrated by one or more of blocks 2102-2116. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 2102, method 2100 includes training, by a first computing device, a first machine learning model using hematology training set data, wherein the first machine learning model is trained to identify one or more blood sample parameters.

At block 2104, method 2100 includes for receiving, by a second computing device, cell data from one or more sensors communicatively coupled to the second computing device.

In some examples, the one or more identifiable parameters associated with blood cells comprises one or more identifiable parameters associated with red blood cells. In some examples, the one or more identifiable parameters associated with red blood cells comprises one or more of the following: (i) total red blood count (RBC), (ii) mean corpuscular volume (MCV), (iii) hemoglobin (HGB), (iv) hematocrit (HCT), (v) mean corpuscular hemoglobin (MCH), (vi) mean corpuscular hemoglobin concentration (MCHC), (vii) red distribution width (RDW), (viii) reticulocyte count (Retic), (ix) percentage of reticulocyte (% Retic), (x) platelet count (PLT), (xi) mean platelet volume (MPV), (xii) plateletcrit (PCT), and (xiii) platelet distribution width (PDW).

In some examples, the one or more identifiable parameters associated with blood cells comprises one or more identifiable parameters associated with white blood cells. In some examples, the one or more identifiable parameters associated with white blood cells comprises one or more of the following: (i) white blood count (WBC), (ii) absolute neutrophil count (NEU), (iii) absolute lymphocyte count (LYM), (iv) absolute monocyte count (MONO), (v) absolute eosinophil count (EOS), (vi) absolute basophil count (BASO), (vii) percentage neutrophils (% NEU), (viii) percentage lymphocytes (% LYM), (ix) percentage monocytes (% MONO), (x) percentage absolute eosinophils (% EOS), and (xi) percentage basophils (% BASO).

In some examples, the first computing device comprises a hematology analyzer. In some examples, the first computing device further comprises a cloud-based modeling computing device. In some examples, the hematology analyzer comprises a flow cytometer.

At block 2106, method 2100 includes, using the first machine learning model and based at least in part on data from on the received cell data, diagnostic data associated with a first portion of the blood sample, wherein the diagnostic data comprises one or more identifiable parameters associated with blood cells.

In examples, determining the diagnostic data comprises evaluating detected cell size and detected cell complexity. In some examples, determining the diagnostic data comprises evaluating detected cell size and detected fluorescence.

At block 2108, method 2100 includes training, by the first computing device, a second machine learning model using image training set data, wherein the second machine learning model is trained to identify one or more attributes associated with a plurality of blood sample cells.

At block 2110, method 2100 includes receiving, by a third computing device, from one or more imaging sensors communicatively coupled to the third computing device, an image of a plurality of cells of a second portion of the blood sample.

In some examples, the third computing device comprises a morphology analyzer. In some examples, the morphology analyzer comprises one or more morphology processors communicatively coupled to the one or more imaging sensors, one or more energy sources optically coupled to the one or more imaging sensors, and an objective lens optically coupled to the one or more imaging sensors. In some examples, if the stain intensity is relatively low, modifying the intensity of the light source includes decreasing the intensity of the light source.

At block 2112, method 2100 includes determining, by the third computing device, using the second machine learning model and based at least in part on the image of the plurality of cells of the second portion of the blood sample, one or more attributes of the plurality of cells.

In some examples, determining the one or more attributes of the plurality of cells comprises identifying a subset of the plurality of cells having a cell size or a cell morphology associated with left shift. In some examples, retraining the first machine learning model using the updated one or more of the identifiable parameters comprises retraining the first machine learning model in response to identifying the subset of the plurality of cells having the cell size of the cell morphology associated with left shift. In some examples, determining the one or more attributes of the plurality of cells comprises identifying a subset of the plurality of cells having a cell size or a cell morphology associated with small pathologic red blood cells. In some examples, retraining the first machine learning model using the updated one or more of the identifiable parameters comprises retraining the first machine learning model in response to identifying the subset of the plurality of cells having the cell size of the cell morphology associated with small pathologic red blood cells. In some examples, determining the one or more attributes of the plurality of cells comprises identifying individual platelet clumps, and in response to identifying the individual platelet clumps, counting a number of individual platelets in the individual platelet clumps. In some examples, retraining the first machine learning model using the updated one or more of the identifiable parameters comprises retraining the first machine learning model based at least in part on the counted number of individual platelets.

At block 2114, method 2100 involves, based on the determined one or more attributes of the plurality of cells, updating, on the second computing device, the one or more of the identifiable parameters.

At block 2116, method 2100 involves, based on the determined one or more attributes of the plurality of cells, retraining, by the first computing device, the first machine learning model using the updated one or more of the identifiable parameters.

In some examples, method 2000 further includes, comprising emitting a beam of energy with an energy source to impinge cells of the first portion of the first portion of the blood sample within a cuvette.

In some examples, determining the one or more attributes of the plurality of cells comprises determining a number of lymphocytes in the second portion of the blood sample, and wherein the method 2100 further includes, wherein training the one or more machine learning models comprises, based on inputting the one or more training images into the machine learning model, (i) predicting, by the one or more machine learning models, an outcome of a determined condition of the one or more training images, (ii) comparing the at least one outcome to the characteristic of the one or more training images, and (iii) adjusting, based on the comparison, the machine learning model.

In some examples, method 2100 further includes transmitting, by the first computing device, a treatment plan based on the updated one or more of the identifiable parameters.

In one aspect, a tangible non-transitory computer-readable medium, having stored thereon program instructions that, when executed by one or more processors, cause the one or more processors to perform a set of operations comprises (i) receiving, by a first computing device, cell data from one or more sensors communicatively coupled to the first computing device; (ii) determining, by the first computing device, via a first machine learning model, and based at least in part on data from on the received cell data, diagnostic data associated with a first portion of the blood sample, wherein the diagnostic data comprises one or more identifiable parameters associated with blood cells, wherein the first machine learning model was trained using hematology training set data, wherein the first machine learning model is trained to identify one or more blood sample parameters; (iii) receiving, by a second computing device, from one or more imaging sensors communicatively coupled to the second computing device, an image of a plurality of cells of a second portion of the blood sample; (iv) determining, by the second computing device, via a second machine learning model and based at least in part on the image of the plurality of cells of the second portion of the blood sample, one or more attributes of the plurality of cells, wherein the second machine learning model was trained using image training set data, wherein the second machine learning model is trained to identify one or more attributes associated with a plurality of blood sample cells; (v) based on the determined one or more attributes of the plurality of cells, updating, by the first computing device, the one or more of the identifiable parameters; and (vi) retraining the first machine learning model using the updated one or more of the identifiable parameters.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter. The singular forms of the articles “a,” “an,” and “the” include plural references unless the context clearly indicates otherwise. For example, the term “a compound” or “at least one compound” can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

What is claimed is:

1. A computer-implemented method for detecting one or more conditions in a blood sample, the computer-implemented method comprising:
receiving, by a first computing device, cell data from one or more sensors communicatively coupled to the first computing device;
determining, by the first computing device, via a first machine learning model, and based at least in part on data from on the received cell data, diagnostic data associated with a first portion of the blood sample, wherein the diagnostic data comprises one or more identifiable parameters associated with blood cells, wherein the first machine learning model was trained using hematology training set data, wherein the first machine learning model is trained to identify one or more blood sample parameters;
receiving, by a second computing device, from one or more imaging sensors communicatively coupled to the second computing device, an image of a plurality of cells of a second portion of the blood sample;
determining, by the second computing device, via a second machine learning model and based at least in part on the image of the plurality of cells of the second portion of the blood sample, one or more attributes of the plurality of cells, wherein the second machine learning model was trained using image training set data, wherein the second machine learning model is trained to identify one or more attributes associated with a plurality of blood sample cells;
based on the determined one or more attributes of the plurality of cells, updating, by the first computing device, the one or more of the identifiable parameters; and
retraining the first machine learning model using the updated one or more of the identifiable parameters.

2. The computer-implemented method of claim 1, wherein the one or more identifiable parameters associated with blood cells comprises one or more identifiable parameters associated with red blood cells.

3. The computer-implemented method of claim 2, wherein the one or more identifiable parameters associated with red blood cells comprises one or more of the following: (i) total red blood count (RBC), (ii) mean corpuscular volume (MCV), (iii) hemoglobin (HGB), (iv) hematocrit (HCT), (v) mean corpuscular hemoglobin (MCH), (vi) mean corpuscular hemoglobin concentration (MCHC), (vii) red distribution width (RDW), (viii) reticulocyte count (Retic), (ix) percentage of reticulocyte (% Retic), (x) platelet count (PLT), (xi) mean platelet volume (MPV), (xii) plateletcrit (PCT), and (xiii) platelet distribution width (PDW).

4. The computer-implemented method of claim 1, wherein the one or more identifiable parameters associated with blood cells comprises one or more identifiable parameters associated with white blood cells.

5. The computer-implemented method of claim 4, wherein the one or more identifiable parameters associated with white blood cells comprises one or more of the following:
(i) white blood count (WBC), (ii) absolute neutrophil count (NEU), (iii) absolute lymphocyte count (LYM), (iv) absolute monocyte count (MONO), (v) absolute eosinophil count (EOS), (vi) absolute basophil count (BASO), (vii) percentage neutrophils (% NEU), (viii) percentage lymphocytes (% LYM), (ix) percentage monocytes (% MONO), (x) percentage absolute eosinophils (% EOS), and (xi) percentage basophils (% BASO).

6. The computer-implemented method of claim 1, wherein the first computing device comprises a hematology analyzer.

7. The computer-implemented method of claim 6, wherein the first computing device further comprises a cloud-based modeling computing device.

8. The computer-implemented method of claim 6, wherein the hematology analyzer comprises a flow cytometer.

9. The computer-implemented method of claim 6, further comprising emitting a beam of energy with an energy source to impinge cells of the first portion of the first portion of the blood sample within a cuvette.

10. The computer-implemented method of claim 1, wherein determining the diagnostic data comprises evaluating detected cell size and detected cell complexity.

11. The computer-implemented method of claim 1, wherein determining the diagnostic data comprises evaluating detected cell size and detected fluorescence.

12. The computer-implemented method of claim 1, wherein the second computing device comprises a morphology analyzer.

13. The computer-implemented method of claim 12, wherein the morphology analyzer comprises one or more morphology processors communicatively coupled to the one or more imaging sensors, one or more energy sources optically coupled to the one or more imaging sensors, and an objective lens optically coupled to the one or more imaging sensors.

14. The computer-implemented method of claim 1, wherein determining the one or more attributes of the plurality of cells comprises identifying a subset of the plurality of cells having a cell size or a cell morphology associated with left shift.

15. The computer-implemented method of claim 14, wherein retraining the first machine learning model using the updated one or more of the identifiable parameters comprises retraining the first machine learning model in response to identifying the subset of the plurality of cells having the cell size of the cell morphology associated with left shift.

16. The computer-implemented method of claim 1, wherein determining the one or more attributes of the plurality of cells comprises identifying a subset of the plurality of cells having a cell size or a cell morphology associated with small pathologic red blood cells.

17. The computer-implemented method of claim 16, wherein retraining the first machine learning model using the updated one or more of the identifiable parameters comprises retraining the first machine learning model in response to identifying the subset of the plurality of cells having the cell size of the cell morphology associated with small pathologic red blood cells.

18. The computer-implemented method of claim 1, wherein determining the one or more attributes of the plurality of cells comprises identifying individual platelet clumps, and in response to identifying the individual platelet clumps, counting a number of individual platelets in the individual platelet clumps.

19. The computer-implemented method of claim 18, wherein retraining the first machine learning model using the updated one or more of the identifiable parameters comprises retraining the first machine learning model based at least in part on the counted number of individual platelets. 20 The computer-implemented method of claim 1, wherein determining the one or more attributes of the plurality of cells comprises determining a number of lymphocytes in the second portion of the blood sample, and wherein the computer-implemented method further comprises:
comparing the determined number of lymphocytes in the second portion of the blood sample to the absolute lymphocyte count or percentage lymphocytes; and
in response to the determined number of lymphocytes and the absolute lymphocyte count or percentage lymphocytes being outside of a configurable threshold, providing a fault indication.

21. The computer-implemented method of claim 1, further comprising transmitting, by the first computing device, a treatment plan based on the updated one or more of the identifiable parameters.

22. A computer-implemented method for detecting one or more conditions in a blood sample, the computer-implemented method comprising:
training, by a first computing device, a first machine learning model using hematology training set data, wherein the first machine learning model is trained to identify one or more blood sample parameters;
receiving, by a second computing device, cell data from one or more sensors communicatively coupled to the second computing device;
determining, by the second computing device, using the first machine learning model and based at least in part on data from on the received cell data, diagnostic data associated with a first portion of the blood sample, wherein the diagnostic data comprises one or more identifiable parameters associated with blood cells;
training, by the first computing device, a second machine learning model using image training set data, wherein the second machine learning model is trained to identify one or more attributes associated with a plurality of blood sample cells;
receiving, by a third computing device, from one or more imaging sensors communicatively coupled to the third computing device, an image of a plurality of cells of a second portion of the blood sample;
determining, by the third computing device, using the second machine learning model and based at least in part on the image of the plurality of cells of the second portion of the blood sample, one or more attributes of the plurality of cells;
based on the determined one or more attributes of the plurality of cells, updating, on the second computing device, the one or more of the identifiable parameters; and
based on the determined one or more attributes of the plurality of cells, retraining, by the first computing device, the first machine learning model using the updated one or more of the identifiable parameters.

23. A tangible, non-transitory computer-readable medium storing instructions that, when executed, cause one or more processors to perform a set of operations comprising:
receiving, by a first computing device, cell data from one or more sensors communicatively coupled to the first computing device;
determining, by the first computing device, via a first machine learning model, and based at least in part on data from on the received cell data, diagnostic data associated with a first portion of a blood sample, wherein the diagnostic data comprises one or more identifiable parameters associated with blood cells, wherein the first machine learning model was trained using hematology training set data, wherein the first machine learning model is trained to identify one or more blood sample parameters;
receiving, by a second computing device, from one or more imaging sensors communicatively coupled to the second computing device, an image of a plurality of cells of a second portion of the blood sample;
determining, by the second computing device, via a second machine learning model and based at least in part on the image of the plurality of cells of the second portion of the blood sample, one or more attributes of the plurality of cells, wherein the second machine learning model was trained using image training set data, wherein the second machine learning model is trained to identify one or more attributes associated with a plurality of blood sample cells;
based on the determined one or more attributes of the plurality of cells, updating, by the first computing device, the one or more of the identifiable parameters; and
retraining the first machine learning model using the updated one or more of the identifiable parameters.
